(19) United States
(12) Patent Application Publication
(10) Pub. No.: US 2025/0297207 A1
(43) Pub. Date: Sep. 25, 2025

(54) SYSTEM AND METHOD USING ARTIFICIAL INTELLIGENCE FOR BIOREACTOR CULTIVATION AND PROCESSING OF BIOLOGICAL MATERIAL

(71) Applicant: Hypha Labs, Inc., Las Vegas, NV (US)

(72) Inventors: John W. Frailey, HENDERSON, NV (US); Jack C. Frailey, HENDERSON, NV (US); A. Stone Douglas, LA JOLLA, CA (US)

(73) Assignee: Hypha Labs, Inc., Las Vegas, NV (US)

(21) Appl. No.: 19/231,501

(22) Filed: Jun. 8, 2025

Related U.S. Application Data
(63) Continuation-in-part of application No. 19/076,872, filed on Mar. 11, 2025.
(60) Provisional application No. 63/563,962, filed on Mar. 12, 2024.
(60) Provisional application No. 63/649,455, filed on May 20, 2024.
(60) Provisional application No. 63/704,493, filed on Oct. 7, 2024.

(51) Int. Cl. C12M 1/36; C12M 1/00; C12M 1/06; C12M 1/12; C12M 1/34

(52) U.S. Cl. CPC C12M 41/48; C12M 27/02; C12M 29/00; C12M 29/20; C12M 37/00; C12M 41/12

(57) ABSTRACT

The embodiments of the present invention disclose a method for cultivating mycelium in a controlled environment including loading sterile substrate into sealed growth chambers and initializing an artificial intelligence (AI)-controlled system to manage environmental parameters. The method involves regulating oxygenation and temperature using sensor feedback, agitating the substrate while delivering nutrients, and continuously monitoring growth conditions. The AI system dynamically adjusts environmental settings based on real-time and historical data to optimize mycelial development. Upon reaching a predetermined growth threshold, the mycelium is harvested and transferred for further processing. The chambers are then sterilized and prepared for the next cultivation cycle. This method enables consistent, scalable production of high-quality mycelial biomass.

### CROSS-REFERENCED TO RELATED APPLICATIONS

This patent application is a continuation-in-part application, and claims benefit of pending U.S. Non Provisional application Ser. No. 19/076,872, entitled “PORTABLE BIOREACTOR FOR MYCELIUM” filed Mar. 11, 2025 by CRAIG ELLINS, and claims the benefit of application of U.S. Provisional application Ser. No. 63/704,493, entitled “PORTABLE BIOREACTOR FOR MYCELIUM” filed Oct. 7, 2024 by CRAIG ELLINS, and claims the benefit of application of U.S. Provisional application Ser. No. 63/649,455, entitled “BIOREACTOR FOR CULTIVATING AND TRANSFORMING MYCELIUM” filed May 20, 2024 by CRAIG ELLINS, and claims the benefit of application of U.S. Provisional application Ser. No. 63/563,962, entitled “BIOREACTOR FOR CULTIVATING AND TRANSFORMING MYCELIUM” filed Mar. 12, 2024 by CRAIG ELLINS, the U.S. patent applications being incorporated herein by reference.

### BACKGROUND

Mushrooms are grown in soil and certain chemical components are extracted for medical and other uses from the harvested mushrooms. Despite the growing interest and demand for mushroom-derived medical products, several challenges and supply constraints persist within the market, impacting availability, accessibility, and quality. Variability in mushroom species, cultivation conditions, extraction techniques, and product formulations can result in inconsistent product quality and efficacy.

### BRIEF DESCRIPTION OF THE DRAWINGS

FIG. 1 shows for illustrative purposes only an example of bioreactor cultivation of mushrooms of one embodiment.

FIG. 2 shows for illustrative purposes only an example of a bioreactor app of one embodiment.

FIG. 3 shows for illustrative purposes only an example of commercial and home installations of one embodiment.

FIG. 4 shows a block diagram of an overview flow chart of the bioreactor cultivation of mushrooms of one embodiment.

FIG. 5 shows a block diagram of an overview of the bioreactor cultivation steps of one embodiment.

FIG. 6 shows for illustrative purposes only an example of a bioreactor cartridge of one embodiment.

FIG. 7A shows for illustrative purposes only an example of a bioreactor cartridge lid of one embodiment.

FIG. 7B shows for illustrative purposes only an example of a bioreactor cartridge magnetic motor support of one embodiment.

FIG. 8 shows for illustrative purposes only an example of a bioreactor cartridge agitator of one embodiment.

FIG. 9 shows for illustrative purposes only an example of a bioreactor cartridge motor driver of one embodiment.

FIG. 10 shows for illustrative purposes only an example of a bioreactor cartridge agitator rotor rod connection of one embodiment.

FIG. 11 shows for illustrative purposes only an example of a bioreactor cartridge heating element base of one embodiment.

FIG. 12 shows for illustrative purposes only an example of a bioreactor cartridge thermistor tube of one embodiment.

FIG. 13 shows for illustrative purposes only an example of a bioreactor system model 2 of one embodiment.

FIG. 14 shows for illustrative purposes only an example of an artificial intelligence (AI) bioreactor cloud control system of one embodiment.

FIG. 15 shows a block diagram of an overview of types of production mycelium of one embodiment.

FIG. 16 shows a block diagram of an overview of types of harvested mycelium bioactive compounds of one embodiment.

FIG. 17 shows a block diagram of an overview of bioreactor control of growth conditions of one embodiment.

FIG. 18 shows a block diagram of an overview of the extraction of mycelium bioactive compounds of one embodiment.

FIG. 19 shows for illustrative purposes only an example of the bioreactor cloud data app of one embodiment.

FIG. 20 shows for illustrative purposes only an example of a magnetic collar of one embodiment.

FIG. 21 shows for illustrative purposes only an example of an agitator rotor rod of one embodiment.

FIG. 22 shows for illustrative purposes only an example of a bioreactor cartridge controls of one embodiment.

FIG. 23 shows for illustrative purposes only an example of a control display screen of one embodiment.

FIG. 24 shows for illustrative purposes only an example of oxygenation air flow vents of one embodiment.

FIG. 25 shows for illustrative purposes only an example of a transparent view of a bioreactor of one embodiment.

FIG. 26A shows for illustrative purposes only an example of mycelium liquid culture-nutrients in a liquid solution of one embodiment.

FIG. 26B shows for illustrative purposes only an example of plating spores to culture of one embodiment.

FIG. 27 shows for illustrative purposes only an example of refillable growing medium cartridge of one embodiment.

FIG. 28A shows for illustrative purposes only an example of a syringe of one embodiment.

FIG. 28B shows for illustrative purposes only an example of syringe extracting liquid nutrient of one embodiment.

FIG. 28C shows for illustrative purposes only an example of syringe injecting best growth spores of one embodiment.

FIG. 29 shows for illustrative purposes only an example of refillable growing medium cartridge placed in bioreactor of one embodiment.

FIG. 30 shows a block diagram of an overview flow chart of the bioreactor cultivation of mushrooms of one embodiment.

FIG. 31A shows for illustrative purposes only an example of refillable growing medium cartridge placed in bioreactor of one embodiment.

FIG. 31B shows for illustrative purposes only an example of a bioreactor app of one embodiment.

FIG. 32 shows a block diagram of an overview flow chart of AI-controlled mycelium cultivation manufacturing of one embodiment.

### SUMMARY OF THE INVENTION

The invention relates to a system and method for cultivating mycelium in a controlled bioreactor environment using an artificial intelligence (AI)-based cultivation system. The system and method involve the use of sealed growth chambers into which a sterile growth substrate is introduced. An AI-controlled system manages environmental parameters within each chamber, including but not limited to oxygenation, temperature, and agitation. Sensor data collected from each chamber is continuously analyzed by the AI system to maintain or modify environmental conditions in response to both real-time and historical data.

The system and method include the regulation of oxygen levels through sterile air delivery and microbubble diffusion, thermal management using heating elements, and substrate agitation to promote uniform distribution of gases, nutrients, and heat. Environmental conditions such as temperature, oxygen concentration, and agitation rates are continuously monitored. The AI system performs adaptive control by modifying one or more parameters based on current sensor feedback and prior cultivation outcomes. The system also incorporates a human-in-the-loop component, allowing for manual input and oversight. Feedback from human operators and results from prior cultivation cycles contribute to the ongoing refinement of the AI's predictive models.

Upon detection that mycelial biomass has reached a predefined growth condition or threshold, harvesting is initiated, followed by sterilization and preparation of the chambers for subsequent cycles. The system and method enable consistent and scalable production of high-quality mycelial material while supporting automated adjustments, user interaction, and continuous learning. The system is designed to support reproducibility and operational efficiency across various applications, including those in biotechnology, pharmaceuticals, and related industries.

In addition to the AI-driven environmental control and mycelium cultivation system and methods, in one embodiment, the system includes a cloud-connected infrastructure that enables enhanced data aggregation, model refinement, and user access. Each bioreactor or cultivation chamber can be wirelessly linked to a centralized cloud platform that stores a growing database of historical cultivation data, including successful harvest parameters across a wide range of environmental conditions and mycelium strains. This centralized repository allows the AI system to compare real-time sensor data from any given chamber to a broader global dataset, thereby improving the predictive accuracy and adaptability of environmental control algorithms over time.

In another embodiment, the present invention also supports integration with user-facing software applications, such as mobile or web-based dashboards, which provide operators with real-time monitoring, system alerts, and actionable insights. Through these interfaces, users may access recommended environmental adjustments, initiate chamber sterilization or harvest cycles, and track historical performance metrics. This feedback loop enhances transparency and enables human-in-the-loop supervision where needed, further supporting model refinement and safety.

In another embodiment, over-the-air (OTA) software updates are included to allow the AI models and control algorithms to be continuously improved without requiring physical access to the hardware as a distributed artificial intelligence framework. These updates may include revised environmental control parameters, bug fixes, and expanded support for additional mycelium strains or cultivation objectives. The OTA capability ensures long-term system viability and adaptability as new data becomes available or cultivation goals evolve. When deployed across a distributed network of devices, such as in home, research, or commercial settings, these features enable a globally-informed cultivation platform where users benefit from the collective learning of all units in operation.

### DETAILED DESCRIPTION OF THE INVENTION

In the following description, reference is made to the accompanying drawings, which form a part hereof, and which are shown by way of illustration a specific example in which the invention may be practiced. It is to be understood that other embodiments may be utilized and structural changes may be made without departing from the scope of the present invention.

It should be noted that the descriptions that follow, for example, in terms of the system and method for bioreactor cultivation and processing of biological material and extraction for medicinal uses, devices, and methods are described for illustrative purposes and the underlying system can apply to any number and multiple types of mushrooms. In one embodiment of the present invention, the system and method for bioreactor cultivation and processing of biological material and extraction for medicinal uses, devices, and methods can be configured using multiple hardware systems for cultivation and extraction. System and method for bioreactor cultivation and processing of biological material and extraction for medicinal uses, devices, and methods can be configured to include a home-sized bioreactor-based cultivation system and large-sized commercial mass-production bioreactor-based cultivation and extraction systems using the present invention.

One of the primary challenges facing the market for mushroom-derived medical products is the lack of standardized extraction methods and quality control measures. Without standardized protocols and rigorous quality assurance processes, ensuring the safety, potency, and reliability of mushroom-derived medical products becomes challenging for manufacturers and consumers alike.

There is a growing body of scientific research supporting the therapeutic potential of mushroom-derived bioactive compounds. The supply chain for mushroom-derived medical products is susceptible to disruptions, vulnerabilities, and seasonality factors. Dependence on wild-harvested mushrooms or limited cultivation capacities for specific mushroom species may result in supply shortages, price fluctuations, and market volatility.

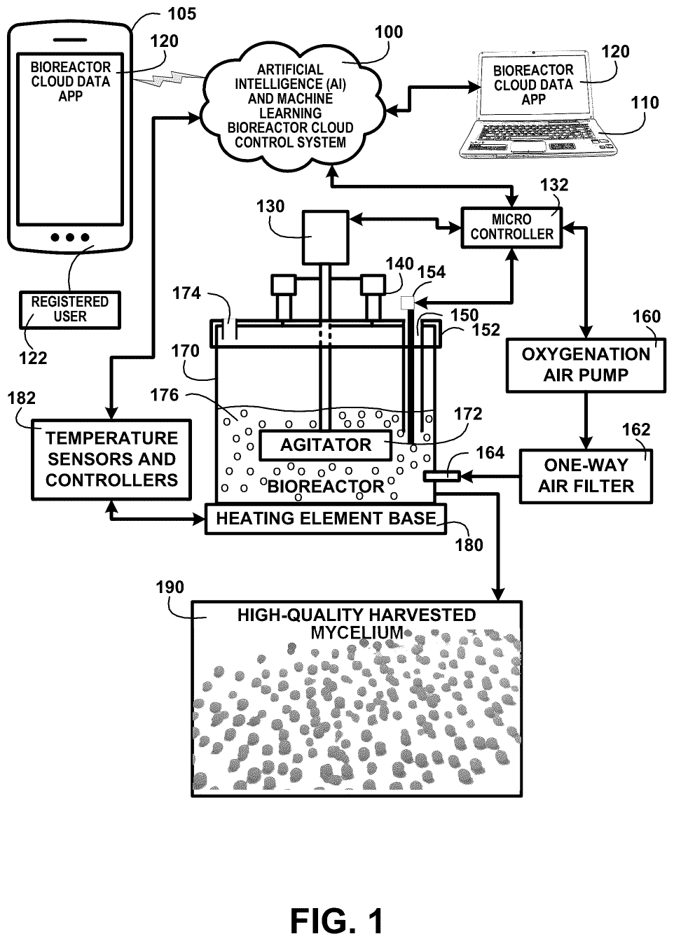

FIG. 1 provides a schematic representation of an embodiment of a bioreactor-based mushroom cultivation system. As depicted, the system includes artificial intelligence (AI) and machine learning ML bioreactor cloud control system 100, which is wirelessly connected to a computer 110 executing a bioreactor cloud data application 120. The bioreactor serves as an engineered platform designed to maintain and control the cultivation conditions of biological organisms under-regulated parameters. A microcontroller 132, integrated with the AI and ML bioreactor cloud control system 100, is configured to manage system functions, including the actuation of a motor driver 130. The motor driver 130 is mechanically linked to a magnetic motor support 140, facilitating precise operational control of internal bioreactor components.

A registered user 122 utilizes a mobile device 105 integrated with the bioreactor cloud data application 120 to remotely interface with the bioreactor cultivation system. This configuration enables the registered user 122 to access, monitor, and review current system settings in real-time. The artificial intelligence bioreactor cloud control system 100 transmits optimized operational parameters to the bioreactor cloud data app 120, providing the registered user 122 with recommended settings. The registered user 122 is further enabled to implement configuration changes through the app 120, facilitating dynamic, remote adjustment of the cultivation environment.

The microcontroller 132 is configured to receive temperature data associated with the cultivation environment from a thermistor 154, which is positioned within a thermistor tube 150 integrated into a lid 152 of the bioreactor cartridge. Additionally, an oxygenation air pump 160 delivers a controlled airflow through a one-way air filter 162 into an oxygenation portal 164, which is structurally integrated into the bioreactor cartridge. This configuration enables regulated oxygen supply to the spores under cultivation, ensuring appropriate atmospheric conditions within the bioreactor.

The bioreactor cartridge 170 is mechanically integrated with an agitator 172, which is operatively coupled to the motor driver 130. The motor driver 130 actuates the agitator 172 to induce rotational movement within the cultivation chamber, thereby enhancing the cultivation process through controlled agitation to promote uniform growth conditions.

A one-way spores injection port 174, integrated into the lid 152 of the bioreactor cartridge 170, enables the sterile introduction of mushroom spores inoculated into a substrate to initiate the controlled-environment cultivation process. Additionally, the bioreactor system is equipped with feed and sampling ports that are functionally coupled to the bioreactor cartridge 170. These ports facilitate the aseptic introduction of nutrient feeds, substrates, or process gases into the cultivation chamber and allow for the extraction of samples for analytical or monitoring purposes, thereby supporting real-time process control and environmental regulation.

The growing medium substrate containing suspended mushroom spores 176 is thermally regulated via a heating element base 180. The microcontroller 132 controls the substrate temperature in response to analytical input data received from the artificial intelligence (AI) and machine learning bioreactor cloud control system 100. Temperature sensors and controllers 182 provide real-time thermal data to the microcontroller 132, enabling precise environmental management. Concurrently, the microcontroller 132 monitors and transmits operational parameters—including agitator rotational speed RPM and internal oxygen concentration levels—to the AI and machine learning bioreactor cloud control system 100. This data is processed to perform dynamic adjustments to component settings, thereby optimizing the cultivation environment. The controlled conditions within the bioreactor significantly enhance the growth rate and quality of the spores, resulting in the production of high-quality harvested mycelium 190 for continuous, year-round cultivation in one embodiment.

The bioreactor system is engineered specifically for the controlled cultivation of mushrooms and is integrated with an artificial intelligence (AI) and machine learning bioreactor cloud control system 100, which is wirelessly connected to a computer running a bioreactor cloud data application 120. A microcontroller 132 interfaces with the AI and machine learning bioreactor cloud control system 100 to manage and regulate the operational parameters of all bioreactor subsystems. The system utilizes AI-generated recommendations to dynamically adjust and maintain optimal oxygenation levels to enhance mycelium development.

The microcontroller activates agitation mechanisms within the growing substrate to promote uniform distribution, ensuring each spore maintains adequate contact with both dissolved nutrients in the liquid substrate and the supplied oxygen. Based on real-time data and historical analysis of successful mycelium cultivations, the AI controller determines the appropriate composition and quantity of nutrients to be added to the substrate. Environmental parameters within the bioreactor are continuously controlled and optimized according to AI-learned growth conditions derived from accumulated cultivation and harvest data.

This intelligent environment modulation facilitates efficient biomass development while selectively reducing the growth of non-target mushroom tissues. This, in turn, simplifies the downstream extraction of bioactive compounds found in the essential medicinal sections of the mushrooms. Moreover, the bioreactor cultivation approach is independent of external environmental conditions and geographic limitations associated with wild harvesting, enabling consistent, year-round, high-yield production of mycelium rich in therapeutic constituents.

The advanced bioreactor cultivation platform integrates artificial intelligence (AI) and machine learning bioreactor cloud control system 100 to enable precision-controlled mushroom cultivation. This system is wirelessly connected to a cloud-based bioreactor cloud data application 120, which facilitates real-time remote monitoring and dynamic control of all bioreactor functions throughout the mycelium growth cycle. A microcontroller 132 serves as the central processing unit, executing AI-driven operational commands, including regulation of oxygenation via an oxygenation air pump 160 and distribution of nutrients within the cultivation substrate. The system delivers AI-optimized oxygen levels and ensures thorough mixing via the agitator 172, which is driven by a motor driver 130, thereby maximizing exposure of each spore within the bioreactor cartridge 170 to both nutrients and oxygen. The AI and machine learning algorithms embedded in the control system 100 continuously analyzes performance data collected across multiple cultivations.

This data is utilized to fine-tune the nutrient profile and adjust operational parameters based on previously identified successful cultivation conditions. The microcontroller 132 monitors input from various sensors, including thermistors 154 and oxygen level detectors, and dynamically adjusts the environmental parameters within the bioreactor to optimize mycelium growth conditions. By focusing growth within a confined and controlled environment, the bioreactor reduces biomass development of non-target mushroom structures, enhancing the efficiency of targeted extraction of high-value medicinal components from the high-quality harvested mycelium 190. Unaffected by external environmental variables or the limitations of wild harvesting, the system supports continuous, year-round, high-output production of potent, medicinal-grade mycelium.

The present invention addresses several limitations associated with conventional mushroom-based medicinal product production methods. By employing a bioreactor cultivation system, the process is decoupled from external environmental variables such as weather conditions and the geographic limitations of wild-grown mushroom harvesting. Traditional methods involving wild harvesting or full-plant cultivation are labor-intensive and yield high volumes of non-essential biomass. In contrast, the bioreactor cartridge 170, under regulation by the microcontroller 132 and the AI and machine learning bioreactor cloud control system 100, facilitates selective cultivation of specific mushroom structures by minimizing the overall fungal biomass. This targeted growth approach significantly reduces biological waste and streamlines the post-harvest extraction process.

Consequently, it enhances the efficiency and precision of isolating the desired medicinal components within the mushroom, ensuring a more sustainable and scalable production method for high-potency therapeutic mycelium.

The cultivation of mushroom mycelium in bioreactors represents a significant advancement in the harnessing of medicinal benefits derived from mushrooms. This innovative methodology, facilitated by artificial intelligence (AI) and machine learning bioreactor cloud control system 100, enables the precise regulation of growth conditions within the bioreactor cartridge 170, leading to optimized mycelium cultivation. Through integration with a microcontroller 132, the bioreactor ensures tight control over critical factors such as temperature monitored by temperature sensors 182, pH, oxygen levels regulated via the oxygenation air pump 160, and nutrient availability adjusted through the AI-driven system, thereby fostering the ideal conditions for mycelial growth.

By leveraging these controlled conditions, the bioreactor optimizes the production of bioactive compounds, significantly enhancing their medicinal potential. The system's ability to regulate environmental factors and nutrient distribution maximizes the mycelium's yield of desired bioactive molecules, while the AI continuously analyzes cultivation success and adjusts operational parameters to further refine the production process. This approach ensures consistent, high-quality bioactive compound extraction, leading to improved medicinal outcomes.

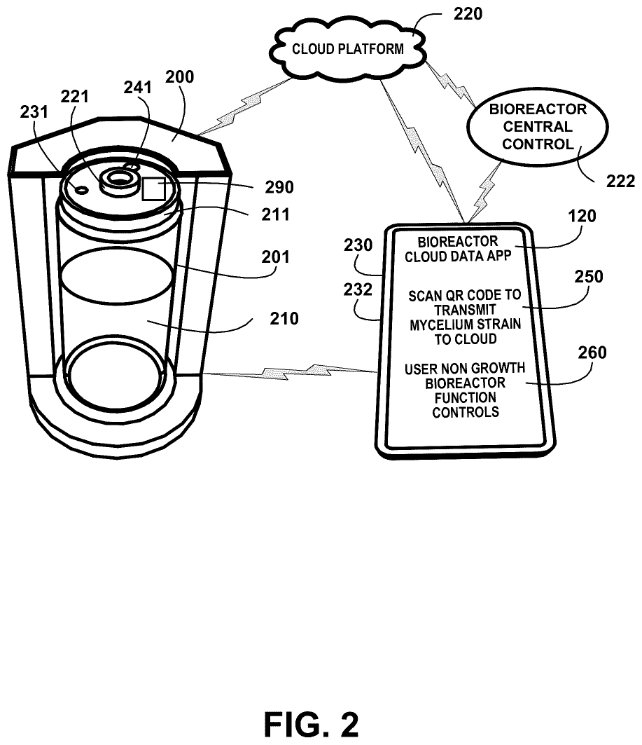

FIG. 2 illustrates an embodiment of a bioreactor application. FIG. 2 depicts the refillable growing medium cartridge 201 installed on a bioreactor 200, which is integrated with an aeration system to prevent spore clumping. Component B of the system is designed to be recyclable, enabling its return for sterilization and refilling. The cartridge cover 211 incorporates a sterile port 221, a nutrient injection port 231, and a pressure relief valve 241 for optimal functionality.

The bioreactor app comprises a user interface dashboard that enables user selection of various sections providing guidance for either a grow or a new recipe. The available sections include APEX 1, WILL1, COSMIC GAZE, SUPERNOVA, and BLACK STAR. APEX 1 allows the user to review monitoring displays for parameters such as temperature, rotation speed, and air agitation. WILL1 provides the user with options for either a manual or a new recipe selection. The manual selection enables the user to configure and modify the set points of the mushroom accelerator at their discretion. In contrast, the new recipe selection allows the app to automatically schedule and implement set point changes for the mushroom accelerator.

The COSMIC GAZE section displays the current actual temperature with a degree reading, rotation speed expressed as a percentage of the maximum 100% rate, and air agitation as a percentage of the 100% rate. The SUPERNOVA section presents the current actual temperature with a degree reading, rotation speed as a percentage of the 100% rate, and air agitation as a percentage of the 100% rate, alongside a graphical chart illustrating changes in set point responses over time.

The SUPERNOVA section includes a subsection labeled ANALYTICS, which displays three key factors: temperature, agitator motor speed, and oxygenation airspeed. The user may select a growth period in days and compare the set point settings to the actual averages of the three factors over the chosen calendar days. This comparison provides the user with insights into whether adjustments to the set points are necessary to more closely align with a recipe. Additionally, the display includes a timeline that highlights when set points have been changed, offering further information on the effectiveness of those particular set-point modifications.

In the ANALYTICS subsection, the user may also modify the set points by using drop-down features to select a desired set point, as well as the specific date and time when the change will take effect. These changes are visually reflected in the chart display, where the color of the chart adjusts to indicate the updated set points.

The SUPERNOVA section includes a subsection labeled ATTRIBUTES. ATTRIBUTES contains a “GROWS” section, which records current cultivation data, including Micropearl Type, Growth Start, Growth End, Started By, and Grow ID. Adjacent to this, historical cultivation data is displayed, providing information such as dates and times, the type of mycelium cultivated, harvest results, and other relevant details from previous cultivations. The settings within this section may be reloaded and rebooted, as necessary.

The bioreactor app further includes a store feature that allows users to order various mycelium species, such as Enoki, Lion's Mane, and Shiitake. The order form within the store displays the prices for each species and includes a purchase cart for managing selections.

A user's smartphone 230, with the bioreactor cloud data app 120 installed, enables the user to interact with the refillable growing medium cartridge 201, the cloud platform 220, and the bioreactor central control 222. The smartphone 230 is used to scan a QR code to transmit the mycelium strain data to the cloud 250. Additionally, the user may utilize the bioreactor cloud data app 120 to control non-growth bioreactor functions 260.

A QR code 290, attached to the cartridge cover, contains information about the mycelium strain. When scanned by the user's smartphone camera 232, the QR code transmits the strain details to the cloud platform 220. The cloud platform 220 subsequently sends control settings for the bioreactor, including agitation rate, temperature, and oxygen flow, to optimize the environment for mycelium growth. The bioreactor cloud data app 120 also allows the user to manage non-growth functions, such as controlling LED light color. The bioreactor central control 222 generates the QR code for identifying the cultured mycelium and sends batch progress updates to the user while making necessary adjustments to the bioreactor as the culture develops.

In one embodiment, a portable and compact bioreactor for controlled cultivation of mycelium spores includes a bioreactor cartridge 170 of FIG. 1 with an injection port 174 of FIG. 1, wherein the bioreactor cartridge 170 of FIG. 1 is removably coupled to the bioreactor 100 of FIG. 1 and contains a sterile substrate 210 comprising organic materials as a nutrient. The injection port 174 of FIG. 1 is a sealed, one-way sterile inlet configured to introduce the mycelium spores and predetermined nutrients into the sterile substrate while preventing contamination.

Further including components, the system comprises a pressure relief valve 241 coupled to the bioreactor 100 of FIG. 1, configured to prevent pressure build-up from heating the sterile substrate 176 of FIG. 1. A bioreactor microcontroller 132 of FIG. 1 is coupled to the bioreactor cartridge 170 of FIG. 1, configured to monitor heat, oxygen, and nutrient levels within the system. A heating element 180 of FIG. 1 is coupled to the bioreactor microcontroller 132 of FIG. 1 and configured to heat the sterile substrate 176 of FIG. 1 within the bioreactor cartridge 170 of FIG. 1 to a predetermined temperature. An air pump 160 of FIG. 1 is coupled to the bioreactor cartridge 170 of FIG. 1 and the bioreactor microcontroller 132 of FIG. 1, configured to supply oxygenation to the sterile substrate 176 of FIG. 1. An agitator 172 of FIG. 1 located within the bioreactor cartridge 170 of FIG. 1 is coupled to a motor driver 130 of FIG. 1, which is in turn coupled to the bioreactor microcontroller 132 of FIG. 1, and is configured to rotate at a predetermined speed to distribute heat, oxygen, and nutrients among the sterile substrate 176 of FIG. 1 with predetermined parameters.

A cloud control system 100 of FIG. 1 is coupled to the bioreactor microcontroller 132 of FIG. 1, configured to analyze empirical data related to the cultivation of the sterile substrate 176 of FIG. 1 and to determine the predetermined speed, predetermined parameters, and predetermined nutrients required to produce specific harvest results. The cloud control system 100 of FIG. 1 is also capable of making future automatic adjustments to the predetermined speed, predetermined parameters, and predetermined nutrients based on the specific harvest results. Additionally, a bioreactor cloud data app 120 of FIG. 1, coupled to a mobile device 230 of the user, is configured to remotely monitor the settings of the bioreactor 100 of FIG. 1, allow the user to adjust the settings, and provide recommended settings based on the analysis from the cloud control system 100 of FIG. 1.

In yet another embodiment, the portable and compact bioreactor for controlled cultivation of mycelium spores is further comprising an artificial intelligence (AI) and machine learning bioreactor cloud control system 100 of FIG. 1 wirelessly coupled to a bioreactor cloud data app 120 of FIG. 1, which is coupled to a user's mobile device 230. The bioreactor cloud data app 120 of FIG. 1 is configured to remotely monitor the settings of the bioreactor 100 of FIG. 1, allow the user to remotely adjust the settings, and receive recommended settings based on the analysis provided by the cloud control system 100 of FIG. 1. The bioreactor cloud data app 120 of FIG. 1 is further configured to allow the user to self-report harvest results to the artificial intelligence (AI) and machine learning bioreactor cloud control system 100 of FIG. 1. The bioreactor microcontroller 132 of FIG. 1 and bioreactor cloud data app 120 of FIG. 1 are configured to receive parameter adjustment settings to automatically regulate parameter settings and optimize cultivation results in the bioreactor cartridge 170 of FIG. 1 based on the analytical input data from the artificial intelligence (AI) and machine learning bioreactor cloud control system 100 of FIG. 1, including user self-reported harvest results.

Empirical data, including cultivation and harvest information, is transmitted automatically from each bioreactor microcontroller 132 of FIG. 1 and the user's mobile device 230 via the bioreactor cloud data app 120 of FIG. 1 to the artificial intelligence (AI) and machine learning bioreactor cloud control system 100 of FIG. 1. The artificial intelligence (AI) and machine learning bioreactor cloud control system 100 of FIG. 1 is further configured to compare past harvest results and past parameter settings to current harvest results and cultivation settings, in order to determine optimal settings during cultivation that produce optimized harvest results, and base recommended adjustments on this comparison. Additionally, the bioreactor 100 of FIG. 1 includes a front pivoting section of a lid 152 of FIG. 1, which is designed to pivot open to facilitate the installation of a bioreactor cartridge 170 of FIG. 1 and make connections to the microcontroller 132 of FIG. 1, air pump 160 of FIG. 1 oxygenation system, agitator drive 172 of FIG. 1, and the heating element 180 of FIG. 1.

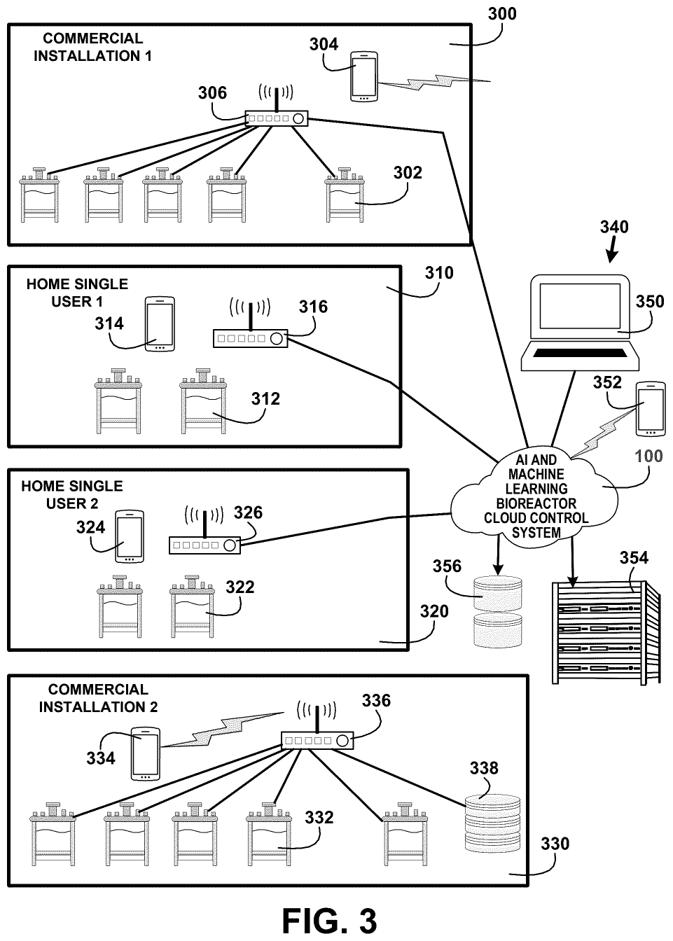

FIG. 3 shows for illustrative purposes only an example of commercial and home installations of one embodiment. FIG. 3 shows bioreactor cultivation in production for consumer home use and commercial industrial use. As shown in commercial installation 1 300, a plurality of bioreactor systems 302 is used to increase the total production of mycelium for the extraction of bioactive compounds for use in medicinal products. The commercial operation includes a microcontroller 306 that is coupled to bioreactors. A user mobile device having a mobile application 304 allows the commercial personnel to be informed of each phase of each of the plurality of bioreactors systems 302 stage of production. At least one microcontroller 132 of FIG. 1 coupled to the plurality of bioreactor systems 302 communicates with a bioreactor cloud interface 340 to regulate conditions in each of the plurality of bioreactor systems 302.

Shown in a home single user 1 310 use are, for example, two bioreactor systems 312. A home user mobile device having a mobile application 314 provides the home user with production data from the microcontroller 316 via the artificial intelligence (AI) and machine learning bioreactor cloud control system 100 to keep the home user current on the growth conditions.

A home single user 2 320 use is shown, for example, having two bioreactor systems 322. A user mobile device having a mobile application 324 provides production condition status to the home user from the microcontroller 326 via the artificial intelligence (AI) and machine learning bioreactor cloud control system 100 that is wirelessly receiving determined regulation of the conditions through a bioreactor cloud interface 340.

Commercial installation 2 330 includes multiple bioreactor systems 332 actively engaged in mycelium cultivation. A user mobile device running a mobile application 334 receives real-time data on growth conditions and regulatory adjustments from a microcontroller 336 operatively connected to the bioreactors. Monitoring data, including environmental and operational parameters, is stored in a plurality of databases 338 for analysis and recordkeeping.

A bioreactor cloud interface 340 coupled to a computer having a mobile application 350 to provide production levels on all bioreactor systems in use. A user mobile device having a mobile application 352 allows central production monitoring personnel to determine the operating conditions of the bioreactor systems. The artificial intelligence (AI) and machine learning bioreactor cloud control system 100 is coupled to a plurality of servers 354 and a plurality of databases 356 of one embodiment.

Manufacturing of the bioreactors is available for large-scale production of medicinal plants in one embodiment for mushrooms. In one embodiment, the bioreactors are scaled up in size and capacity to allow higher production of the mushroom components to meet the pharmaceutical demand for the mushroom medicinal products.

Bioactive compound production from mushroom medicinal plants contains a myriad of bioactive compounds with therapeutic properties, including alkaloids, flavonoids, terpenoids, and polyphenols. Bioreactors can be engineered to stimulate the biosynthesis of specific secondary metabolites through elicitation, precursor feeding, or genetic engineering techniques, thereby enhancing the production of target compounds for pharmaceutical or nutraceutical applications.

The scalability and efficiency of the bioreactors offer scalability and efficiency advantages over traditional cultivation methods, allowing for higher plant densities, reduced land and water requirements, and increased productivity per unit area. This enables cost-effective large-scale production of medicinal plants with minimal environmental impact and resource utilization.

The extraction processes of mushroom components harness their therapeutic potential for applications, including pharmaceuticals, nutraceuticals, cosmetics, and functional foods. The diverse extraction processes employed to isolate mushroom components are used for the specific bioactive compounds and differing types of mushrooms.

Several extraction methods are utilized to isolate bioactive compounds from mushrooms, each offering unique advantages in terms of efficiency, selectivity, and scalability. Some methods include solvent extraction. Solvent extraction is one of the most widely used methods for isolating mushroom components. It involves the use of organic solvents such as ethanol, methanol, acetone, or water to dissolve and extract target compounds from mushroom biomass. Solvent extraction can be performed using conventional methods such as maceration, percolation, or Soxhlet extraction, as well as ultrasound-assisted extraction UAE and supercritical fluid extraction SFE.

Supercritical fluid extraction SFE is a sophisticated extraction technique that utilizes supercritical fluids such as carbon dioxide CO₂ as solvents. Under high pressure and temperature conditions, CO₂ exhibits both gas-like and liquid-like properties, enabling efficient extraction of non-polar and semi-polar compounds from mushrooms. SFE offers advantages such as high selectivity, minimal solvent residue, and reduced environmental impact, making it particularly suitable for extracting heat-sensitive or thermally labile bioactive compounds.

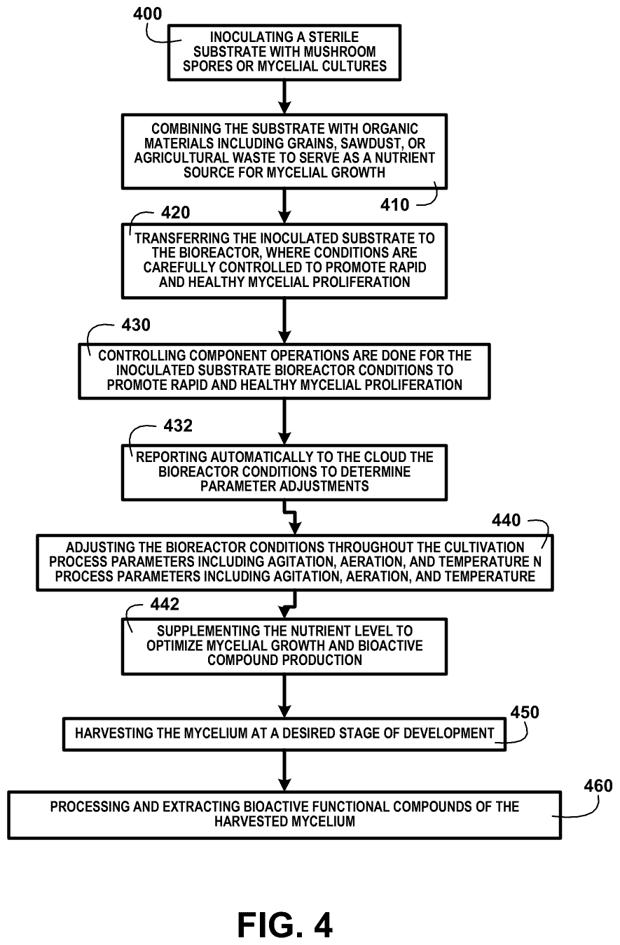

FIG. 4 shows a block diagram of an overview flow chart of the bioreactor cultivation of mushrooms of one embodiment. FIG. 4 shows inoculating a sterile substrate with mushroom spores or mycelial cultures 400. The mushroom spores are fed nutrients by combining the substrate with organic materials including grains, sawdust, or agricultural waste to serve as a nutrient source for mycelial growth 410.

Reaching a predetermined growth the process continues by transferring the inoculated substrate to the bioreactor, where conditions are carefully controlled to promote rapid and healthy mycelial proliferation 420. Continual monitoring and controlling component operations are done for the inoculated substrate bioreactor conditions to promote rapid and healthy mycelial proliferation 430. The control includes reporting automatically to the cloud the bioreactor conditions to determine parameter adjustments 432.

Adjusting the bioreactor conditions throughout the cultivation process parameters including agitation, aeration, and temperature 440 improves cultivation results. The spores grow well by supplementing the nutrient level to optimize mycelial growth and bioactive compound production 442. The controlled bioreactor cultivation results in harvesting the mycelium at a desired stage of development 450. Harvesting is followed by processing and extracting bioactive functional compounds of the harvested mycelium 460 of one embodiment.

The bioactive compounds extracted from the bioreactor cultured mycelium include Polysaccharides. Mushroom mycelium cultivated in bioreactors is rich in polysaccharides, and complex carbohydrates with diverse physiological effects. Beta-glucans, a type of polysaccharide found in many mushroom species, exhibit immunomodulatory properties and have been studied for their potential to boost immune function and combat diseases.

Cultivating mushroom mycelium in the bioreactors represents an innovative approach to harnessing nature's pharmacy and unlocking the therapeutic potential of bioactive compounds. By optimizing conditions for mycelial growth and extraction the production of high-quality extracts rich in polysaccharides, triterpenoids, and other valuable compounds is increased in volume and quality. The optimization includes proprietary formulation of bioreactor substrates including nutrients formulated to promote the cultured mushroom mycelium growth and quality. Additionally, optimization includes proprietary formulation of extraction liquids to increase the quantity and quality of the extracted bioactive compounds.

These bioactive compounds offer a wide range of medicinal benefits, from immune modulation and anti-inflammatory effects to antioxidant activity. The bioreactor technology of this invention improves the development of novel therapeutics and nutraceuticals derived from cultured mushroom mycelium.

Mushroom Mycelium is cultured in the bioreactors as a source of medicinal bioactive compounds. Mushroom mycelium, the intricate network of thread-like structures that form the vegetative part of fungi, holds remarkable potential as a source of bioactive compounds with medicinal benefits. Mycelium, often referred to as the “hidden half” of fungi, plays a crucial role in nutrient cycling, soil health, and ecosystem resilience. Beyond its ecological significance, mycelium harbors a treasure trove of bioactive molecules that have been studied for their therapeutic properties.

Bioreactor culture mushroom mycelium represents a natural source of medicinal products, offering a rich source of bioactive compounds with diverse medicinal properties. From immune support and anti-inflammatory effects to antioxidant activity benefits, bioreactor cultures of mycelium-derived compounds hold promise for addressing a wide range of health concerns. The therapeutic potential of mushroom mycelium may lead to the development of novel treatments and supplements that promote wellness and vitality.

Mycelium, as the vegetative part of fungi, is indeed a versatile and functional organism with numerous applications, particularly in the realm of health and wellness.

Medicinal Properties: Mycelium produces a wide array of bioactive compounds, including polysaccharides, terpenoids, and phenolic compounds, which have been studied for their medicinal properties. These compounds exhibit biological activities such as anti-inflammatory, antioxidant, immunomodulatory, and antimicrobial effects.

Nutritional Benefits: Some species of mushrooms grown from mycelium are not only delicious but also highly nutritious. They are rich sources of protein, dietary fiber, vitamins such as B vitamins and vitamin D, and minerals such as selenium and potassium. Incorporating these mushrooms into diets can help improve overall nutrition and contribute to better health outcomes. Gut Health: Mycelium-derived products, particularly those containing prebiotic fibers, can support gut health by promoting the growth of beneficial gut bacteria. These fibers serve as food for probiotics, helping to maintain a healthy balance of microorganisms in the gut microbiome. A balanced gut microbiome is associated with improved digestion, enhanced immune function, and reduced risk of gastrointestinal disorders.

Mushroom Mycelium is cultured in the bioreactors as a source of medicinal bioactive compounds. Mushroom mycelium, the intricate network of thread-like structures that form the vegetative part of fungi, holds remarkable potential as a source of bioactive compounds with medicinal benefits. Beyond its ecological significance, mycelium harbors bioactive molecules that have been studied for their therapeutic properties and the potential medicinal benefits they offer.

Bioactive Compounds Found in Mushroom Mycelium include Beta-Glucans: Beta-glucans are polysaccharides found abundantly in mushroom mycelium. These compounds possess immunomodulatory properties, stimulating the activity of macrophages, natural killer cells, and other components of the immune system. Beta-glucans have been studied for their potential to enhance immune function and combat diseases, including cancer, infections, and autoimmune disorders.

Polysaccharide Peptides: Certain mushroom mycelium species produce polysaccharide peptides, complex molecules with potent antioxidant and anti-inflammatory properties. These compounds have shown promise in protecting cells from oxidative damage, reducing inflammation, and supporting overall health and well-being.

Triterpenoids: Triterpenoids are another class of bioactive compounds found in mushroom mycelium. These molecules exhibit diverse pharmacological activities, including anti-inflammatory, antimicrobial, and anti-cancer properties. Triterpenoids have been studied for their potential to manage chronic inflammatory conditions, prevent infections, and inhibit tumor growth.

Aromatic Compounds: Mushroom mycelium is rich in aromatic compounds, such as phenols and flavonoids, which contribute to its distinctive aroma and flavor. These compounds possess antioxidant properties and may help protect cells from oxidative stress and DNA damage. Additionally, some aromatic compounds found in mycelium have demonstrated antimicrobial activity against pathogens.

Medicinal benefits of mycelium-derived bioactive compounds include immune support with Beta-glucans and other immunomodulatory compounds found in mushroom mycelium have been shown to enhance immune function. By activating immune cells and promoting cytokine production, these compounds help the body mount a robust immune response against infections, tumors, and other threats.

Anti-Inflammatory Effects: Polysaccharide peptides and triterpenoids present in mushroom mycelium exhibit potent anti-inflammatory properties. These compounds help mitigate inflammation by suppressing the production of pro-inflammatory cytokines and inhibiting inflammatory pathways. As a result, mycelium-derived bioactive compounds may benefit individuals suffering from chronic inflammatory conditions such as arthritis, inflammatory bowel disease, and asthma.

Antioxidant Activity: Aromatic compounds and polysaccharide peptides found in mushroom mycelium possess significant antioxidant activity. By scavenging free radicals and reducing oxidative stress, these compounds help protect cells from damage and slow down the aging process. Antioxidant-rich mycelium extracts may therefore contribute to overall health and longevity.

Bioreactor culture mushroom mycelium represents a natural source of medicinal products, offering a rich source of bioactive compounds with diverse medicinal properties. From immune support and anti-inflammatory effects to antioxidant activity benefits, bioreactor cultures of mycelium-derived compounds hold promise for addressing a wide range of health concerns. The therapeutic potential of mushroom mycelium may lead to the development of novel treatments and supplements that promote wellness and vitality.

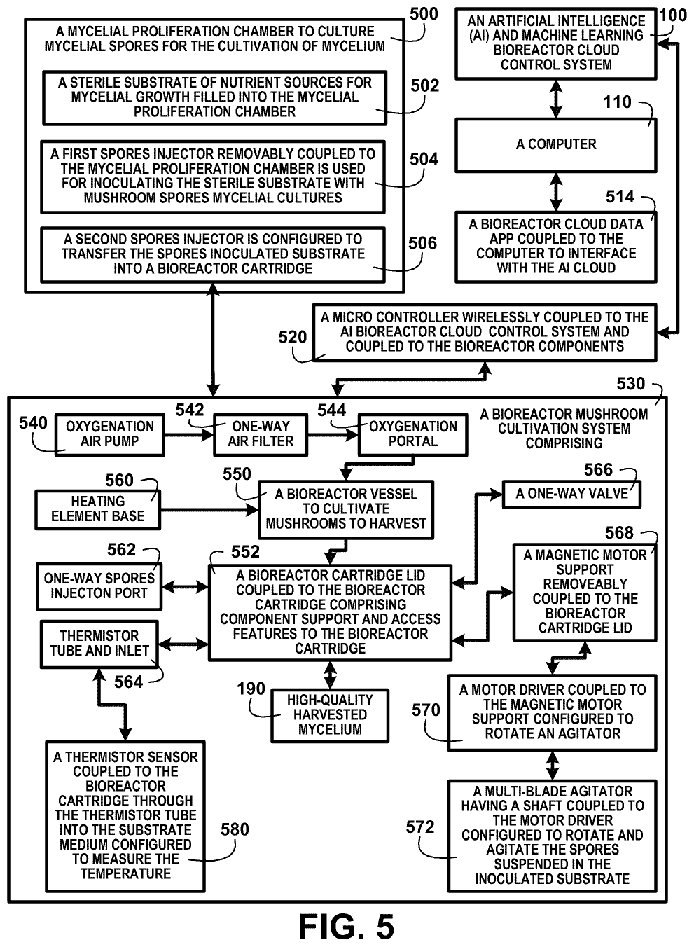

FIG. 5 shows a block diagram of an overview of the bioreactor cultivation steps of one embodiment. FIG. 5 shows a mycelial proliferation chamber to culture mycelial spores for the cultivation of mycelium 500. The cultivation includes a sterile substrate of nutrient sources for mycelial growth filled into the mycelial proliferation chamber 502. A first spores injector removably coupled to the mycelial proliferation chamber is used for inoculating the sterile substrate with mushroom spores mycelial cultures 504. A second spores injector is configured to transfer the spores inoculated substrate into a bioreactor cartridge 506.

An artificial intelligence (AI) and machine learning bioreactor cloud control system 100 receive operational data from various bioreactor cartridge components. A computer 110 is wirelessly connected to the AI system 100, enabling visualization of AI-generated data to assist users in monitoring and evaluating cultivation progress. A bioreactor cloud data application 120 of FIG. 1, coupled to the computer 110, provides an interface to the AI cloud system 514. A microcontroller 132 of FIG. 1 wirelessly coupled to the artificial intelligence (AI) and machine learning bioreactor cloud control system 100 and coupled to the bioreactor components 520 to transmit and receive data and instructions bidirectionally. The microcontroller 132 of FIG. 1 is coupled to the cultivation parameters components to regulate the operations.

A bioreactor mushroom cultivation system comprising 530 an oxygenation air pump 540, one-way air filter 542, oxygenation portal 544, and a bioreactor vessel to cultivate mushrooms to harvest 550.

The microcontroller 132 of FIG. 1 enables bidirectional communication of operational data and control instructions. The microcontroller 132 of FIG. 1 also interfaces with the cultivation parameter control components to regulate bioreactor functions in real-time.

A bioreactor cartridge lid coupled to the bioreactor cartridge comprising component support and access features to the bioreactor cartridge 552 and a vent that is a one-way valve 566. Heat is generated for the spores with a heating element base 560 the bioreactor cartridge sets on. A one-way spores injection port 562 integrated into the lid provides access for the second spores injector to transfer the spores inoculated substrate to be deposited into the bioreactor cartridge.

The thermistor 154 of FIG. 1 is inserted into the substrate through a thermistor tube and inlet 564 and is integrated into the lid. A thermistor sensor coupled to the bioreactor cartridge through the thermistor tube into the substrate medium configured to measure the temperature 580 and transmit the temperature reading to the microcontroller 132 of FIG. 1. The thermistor 154 of FIG. 1 measures the temperature in the bioreactor cartridge.

Magnetic motor support is removably coupled to the bioreactor cartridge lid 568 supports a motor driver coupled to the magnetic motor support configured to rotate an agitator 570. A multi-blade agitator having a shaft coupled to the motor driver configured to rotate and agitate the spores suspended in the inoculated substrate 572. The controlled environment created by the bioreactor cartridge and component allows improved cultivation of a plurality of mushroom species. The controlled environment assures high-quality harvested mycelium 190 of predeterminable quality of one embodiment.

The bioactive compounds extracted from the bioreactor cultured mycelium include Polysaccharides. Mushroom mycelium cultivated in bioreactors is rich in polysaccharides, and complex carbohydrates with diverse physiological effects. Beta-glucans, a type of polysaccharide found in many mushroom species, exhibit immunomodulatory properties, and have been studied for their potential to boost immune function and combat diseases of one embodiment.

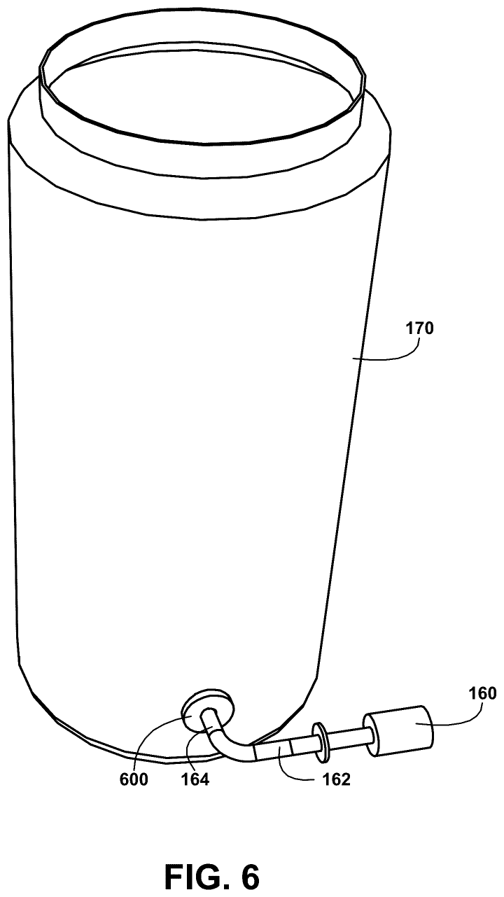

FIG. 6 shows for illustrative purposes only an example of a bioreactor cartridge of one embodiment. FIG. 6 shows that bioreactor cartridge 170, is made of at least one material from a group of at least stainless steel, glass, or plastic and comes in assorted sizes ranging from laboratory-scale to industrial-scale reactors. Oxygenation promotes the growth of the spores. An oxygenation air pump 160 is regulated to supply oxygen through the one-way air filter 162 to purify the airflow. The airflow enters the bioreactor chamber through the oxygenation portal 164 sealed with a gasket 600 of one embodiment.

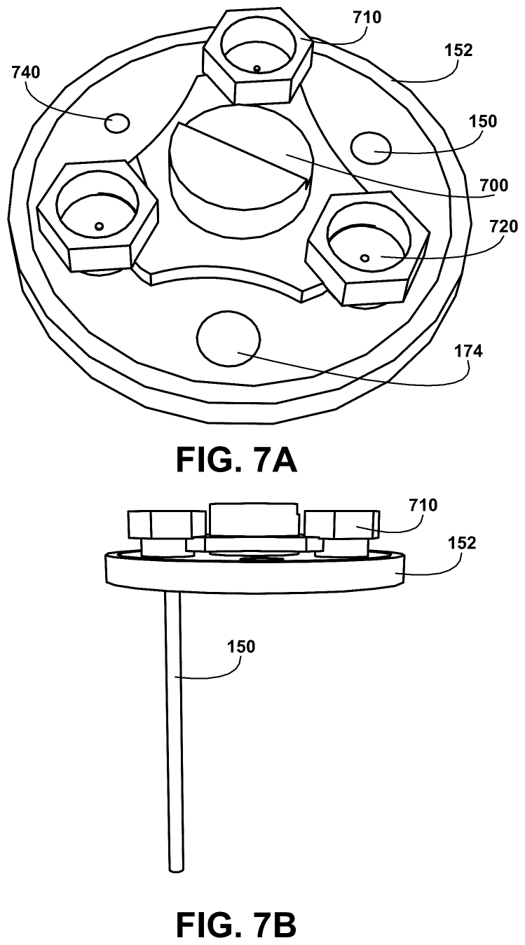

FIG. 7A shows for illustrative purposes only an example of a bioreactor cartridge lid of one embodiment. FIG. 7A shows the lid 152 of the bioreactor cartridge 170 of FIG. 1. The magnetic motor support 140 of FIG. 1 is seated into the couplings 710 and integrated into the lid 152. The couplings 710 includes a magnet 720 to secure the magnetic motor support 140 of FIG. 1 to the lid 152. A drive motor coupling 700 mates with the motor driver 130 of FIG. 1 axle that has double bearings. The thermistor tube 150 extends from a lid inlet downward into the cartridge to allow the thermistor 154 of FIG. 1 to reach into the substrate-growing solution to monitor the temperature. Integrated into the lid 152 is the one-way spores injection port 174 providing access to the cartridge for depositing the substrate and spores. A one-way valve 740 integrated into the lid 152 provides a pressure relief outlet to prevent over-pressuring the bioreactor cartridge of one embodiment.

FIG. 7B shows for illustrative purposes only an example of a bioreactor cartridge magnetic motor support of one embodiment. FIG. 7B shows the lid 152 in a side view with the couplings 710. Also showing is the thermistor tube 150 extending downward from the lid 152 to allow the inserted thermistor 154 of FIG. 1 terminus to be immersed into the substrate-growing medium of one embodiment.

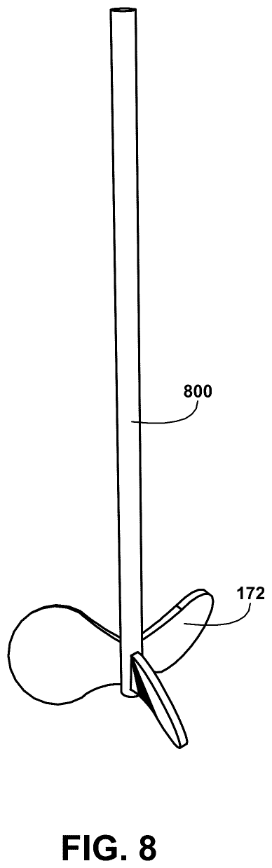

FIG. 8 shows for illustrative purposes only an example of a bioreactor cartridge agitator of one embodiment. FIG. 8 shows the agitator 172 coupled to the agitator rotor rod 800. The agitator rotor rod 800 couples to the motor driver 130 of FIG. 1 axle connector to rotate the agitator 172 to provide agitation to the substrate and spores to provide a uniform mix of the nutrients, oxygen, and temperature to all of the spores. Monitoring of the conditions is performed to make adjustments in rotational speed to maintain optimized growing conditions of one embodiment.

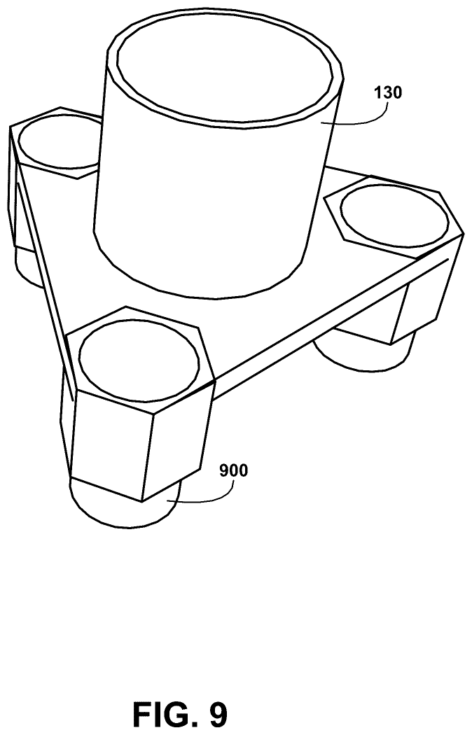

FIG. 9 shows for illustrative purposes only an example of a bioreactor cartridge motor driver of one embodiment. FIG. 9 shows the motor driver 130 coupled to the magnetic support 900. The motor driver 130 is coupled to the microcontroller 132 of FIG. 1 for a power connection and rpm regulation. The magnetic support 900 couples to the lid 152 of FIG. 1 and magnet 720 of FIG. 7A in the couplings 710 of FIG. 7A to secure the motor driver 130 of FIG. 1 in a stable connection of one embodiment.

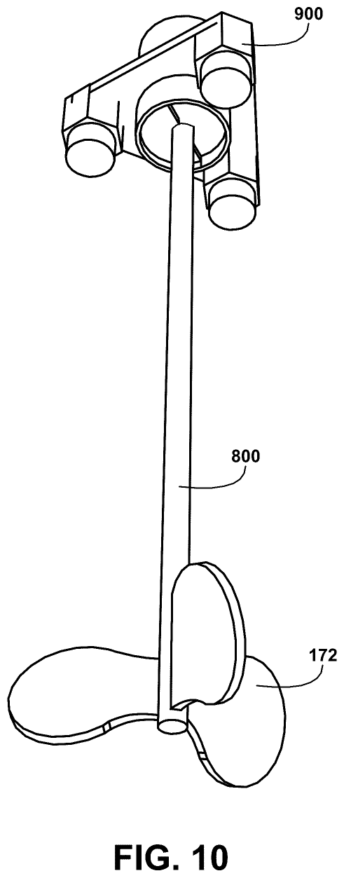

FIG. 10 shows for illustrative purposes only an example of a bioreactor cartridge agitator rotor rod connection of one embodiment. FIG. 10 shows the magnetic support 900 and agitator rotor rod 800 coupled to the motor driver 130 of FIG. 1. The motor driver 130 of FIG. 1 rotates the agitator rotor rod 800 at an rpm regulated by the microcontroller 132 of FIG. 1. The agitator 172 rotated by the agitator rotor rod 800 is continuously mixing the substrate to assure all of the suspended spores are provided nutrients, are fully oxygenated, and kept at an optimal temperature. The agitator 172 fluid movement does not harm the mycelium and the liquid movement also imparts the mycelium marble shapes of one embodiment.

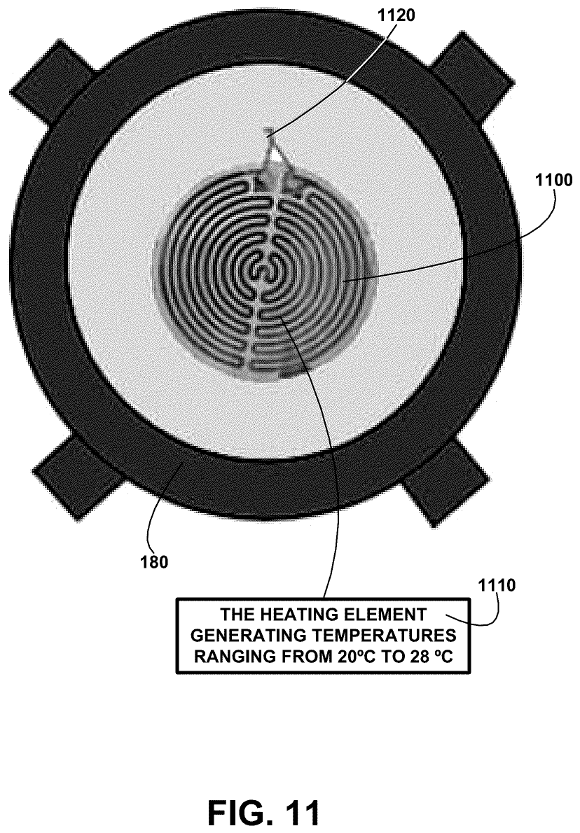

FIG. 11 illustrates an example embodiment of a bioreactor cartridge heating element base. The bioreactor cartridge 170 of FIG. 1 is positioned on the heating element base 180 to maintain optimal growth temperatures. The base includes an outer support ring housing a heating element 1100, which generates controlled temperatures ranging from approximately 20° C. to 28° C., as indicated at 1110. The heating element 1100 is connected to control and power conductors 1120, enabling it to receive power and execute temperature settings issued by the microcontroller 132 of FIG. 1. These settings are determined by the artificial intelligence (AI) and machine learning bioreactor cloud control system 100 of FIG. 1. In one embodiment, the temperature is maintained at approximately 28° C. during the primary growth phase and is subsequently reduced to between 25° C. and 20° C. after the fifth day to enhance Psilocybin production.

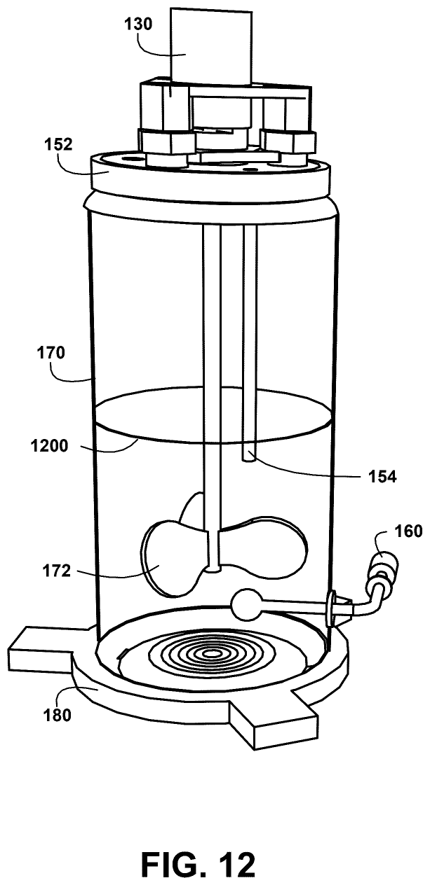

FIG. 12 shows for illustrative purposes only an example of a bioreactor cartridge thermistor tube of one embodiment. FIG. 12 shows the assembled bioreactor cartridge 170 including the motor driver 130, lid 152, oxygenation air pump 160, and heating element base 180. FIG. 12 also shows the interior of the bioreactor cartridge 170 is a substrate level 1200 into which the thermistor tube 150 of FIG. 1 is extended for inserting the thermistor 154 to measure the temperature. The agitator 172 is shown immersed in the substrate to mix the substrate for uniformity of the contents including supplemental nutrients when they are added to the substrate, temperature, and oxygen levels of one embodiment.

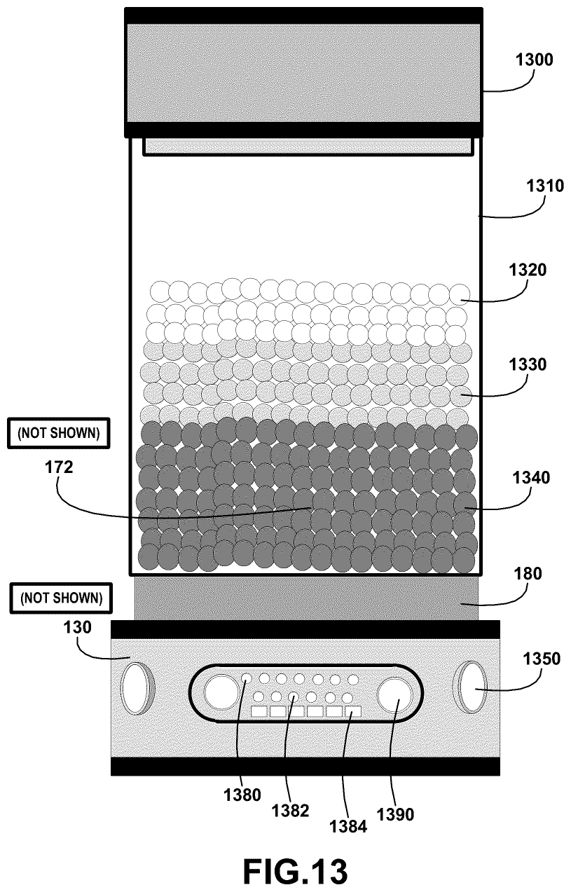

FIG. 13 shows for illustrative purposes only an example of a bioreactor system model 2 of one embodiment. FIG. 13 shows bioreactor system model 2 including a lid cover 1300, and a bioreactor 1310 chamber. The bioreactor 1310 chamber contains oxygenation bubbles 1320, substrate 1330 in bubbles, and mycelium 1340 shaped in pellets by the agitator motion. Bioreactor system model 2 also includes an agitator not shown 172, a heating element base 180, air pump inlet 1350 to draw air into the air pump, a motor driver not shown 130 to rotate the agitator, LED status lights 1380 to inform the user of the operational status, system settings status 1382 illuminated buttons, operation buttons 1384 to show red, yellow, and green signals of component status, and a motor driver on/off 1390 switch of one embodiment.

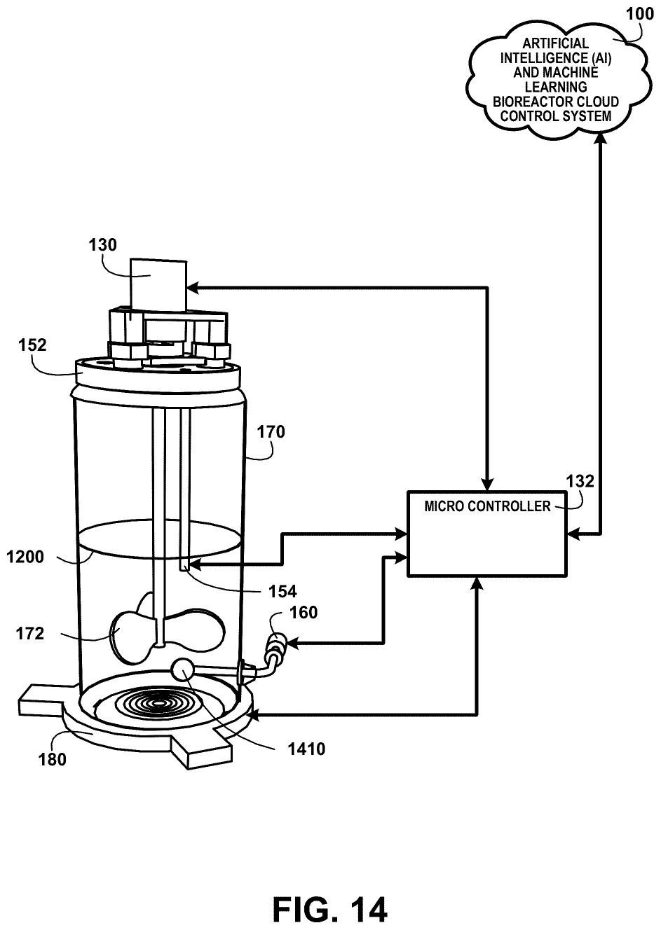

FIG. 14 shows for illustrative purposes only an example of an artificial intelligence (AI) bioreactor cloud control system of one embodiment. FIG. 14 shows the assembled bioreactor cartridge 170 including the motor driver 130, lid 152, and oxygenation air pump 160 through an air stone 1410 of FIG. 14 to create microbubbles ranging from 0.5 μm to 5 μm, and a heating element base 180. FIG. 14 also shows the interior of the bioreactor cartridge 170 is a substrate level 1200 into which the thermistor tube 150 of FIG. 1 is extended for inserting the thermistor 154 to measure the temperature. The agitator 172 is shown immersed in the substrate.

The assembled bioreactor cartridge 170 is coupled to the microcontroller 132 which is wirelessly coupled to the artificial intelligence (AI) and machine learning bioreactor cloud control system 100. The bioreactor conditions monitoring data received by the microcontroller 132 is transmitted to the artificial intelligence (AI) and machine learning bioreactor cloud control system 100. The artificial intelligence (AI) and machine learning bioreactor cloud control system 100 analyzes the data to compare to successful previous cultivations to determine any adjustments to regulate temperature and other growing conditions to optimize the production of one embodiment.

The agitation and mixing systems include agitation mechanisms, including impellers, stirrers, or spargers for mixing nutrients, maintaining homogeneity, and preventing cell settling. The design considerations include impeller geometry, placement, and speed control to optimize mixing efficiency and minimize shear stress on cells.

The temperature control systems include precision temperature controllers for maintaining optimal growth conditions and metabolic activity. Bioreactors utilize jacketed cartridges, external heat exchangers, or immersion heaters coupled with temperature sensors and controllers to achieve precise temperature regulation. The pH and dissolved oxygen monitoring are utilized for monitoring and control of pH and dissolved oxygen levels are critical for cell viability and product yield. Bioreactors are equipped with sensors and control systems to measure and adjust pH using acid or base addition, as well as regulate oxygen concentrations through aeration or agitation speed modulation.

Quality control during manufacture is maintained to ensure the bioreactors are completed with sustainable quality to allow for proper sterilization and aseptic techniques during use without damaging the equipment and ensure sterilization and aseptic quality levels are maintainable to prevent contamination.

Automation and control systems are integrated into the advanced bioreactor manufacture. The automation and control systems, including programmable logic controllers PLCs and supervisory control and data acquisition SCADA systems, monitor and regulate process parameters, automate feeding and sampling operations, and ensure reproducibility and consistency in production.

These bioreactors and related cultivation and extraction equipment are manufactured to provide year-round cultivation. The bioreactors enable year-round cultivation of medicinal plants in a controlled environment, independent of seasonal variations and geographical constraints. This allows for continuous production and a reliable supply of plant-derived bioactive compounds, regardless of climate or location.

The bioreactor systems optimize growth conditions by enabling precise control over key parameters, including temperature, humidity, light exposure, and nutrient delivery. This controlled environment promotes ideal conditions for plant growth, development, and metabolite synthesis, resulting in increased yields, enhanced potency, and consistent quality of medicinal plant extracts.

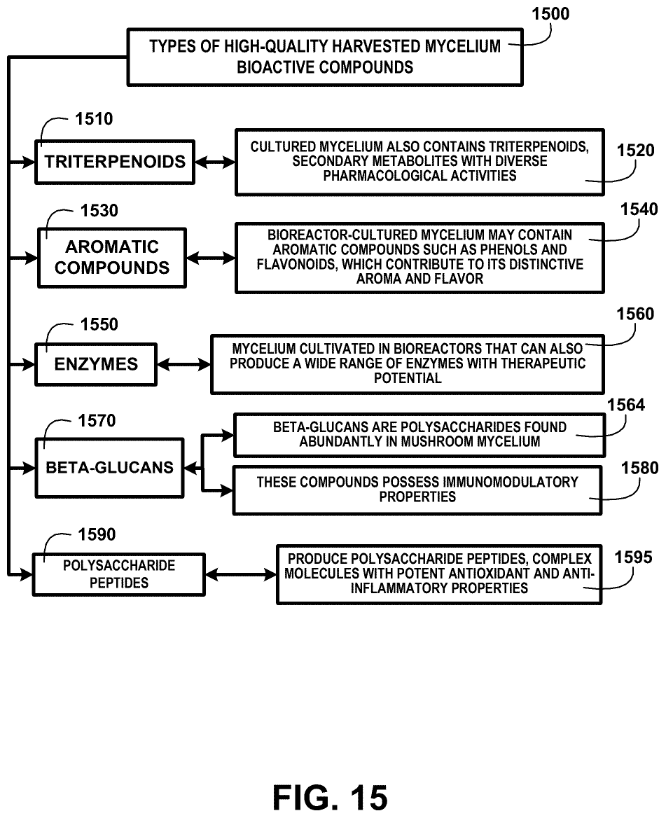

FIG. 15 shows a block diagram of an overview of types of production mycelium of one embodiment. FIG. 15 displays several types of high-quality harvested mycelium bioactive compounds 1500 extracted for use in medicinal products. The mycelium bioactive compounds include triterpenoids 1510. The cultured mycelium also contains triterpenoids, secondary metabolites with diverse pharmacological activities 1520. Another type of mycelium bioactive compound is an aromatic compound 1530. Aromatic compounds 1530 of the bioreactor-cultured mycelium may contain aromatic compounds such as phenols and flavonoids, which contribute to its distinctive aroma and flavor 1540.

Enzymes 1550 are bioactive compounds of the mycelium cultivated in bioreactors that can also produce a wide range of enzymes with therapeutic potential 1560. Beta-glucans 1570 are also extracted from the mycelium cultivated in bioreactors. Beta-glucans are polysaccharides found abundantly in mushroom mycelium 1564. These compounds possess immunomodulatory properties 1580.

Mycelium cultivated in bioreactors also provides extraction of polysaccharide peptides 1590. The extractions from the mycelium cultivated in bioreactors also produce polysaccharide peptides, complex molecules with potent antioxidant and anti-inflammatory properties 1595 of one embodiment.

Triterpenoids: Cultured mushroom mycelium also contains triterpenoids, secondary metabolites with diverse pharmacological activities. These compounds exhibit anti-inflammatory, antioxidant, and anti-cancer properties, making them valuable candidates for therapeutic applications.

Aromatic Compounds: Bioreactor-cultured mycelium may contain aromatic compounds such as phenols and flavonoids, which contribute to their distinctive aroma and flavor. These compounds possess antioxidant properties and may help protect cells from oxidative stress and damage.

Enzymes: Mycelium cultivated in bioreactors can also produce a wide range of enzymes with therapeutic potential. For example, certain mushroom species are known to produce enzymes such as laccase and peroxidase, which have applications in bioremediation, food processing, and pharmaceutical manufacturing.

Medicinal benefits of bioactive compounds from bioreactor cultured mycelium include Immune Modulation. Polysaccharides extracted from cultured mycelium have been shown to modulate the immune system, enhancing the activity of immune cells, and promoting immune surveillance against pathogens. These compounds may help bolster the body's defenses and support overall health and well-being.

Anti-inflammatory effects from triterpenoids found in the bioreactor cultured mycelium exhibit potent anti-inflammatory properties, which may help alleviate symptoms of chronic inflammatory conditions such as arthritis, inflammatory bowel disease, and asthma. By suppressing inflammatory pathways and reducing oxidative stress, these compounds contribute to a balanced immune response and tissue homeostasis. Antioxidant activity is derived from aromatic compounds and polysaccharides extracted from the bioreactor cultured mycelium possess significant antioxidant activity, scavenging free radicals and protecting cells from oxidative damage. These compounds help mitigate oxidative stress and may play a role in preventing age-related diseases and promoting longevity.

By optimizing conditions for mycelial growth and extraction the production of high-quality extracts rich in polysaccharides, triterpenoids, and other valuable compounds is increased in volume and quality. Additionally, optimization includes proprietary formulation of extraction liquids to increase the quantity and quality of the extracted bioactive compounds. These bioactive compounds offer a wide range of medicinal benefits, from immune modulation and anti-inflammatory effects to antioxidant activity.

Mushroom Mycelium is cultured in the bioreactors as a source of medicinal bioactive compounds. Mushroom mycelium, the intricate network of thread-like structures that form the vegetative part of fungi, holds remarkable potential as a source of bioactive compounds with medicinal benefits.

Bioactive Compounds Found in Mushroom Mycelium include Beta-Glucans: Beta-glucans are polysaccharides found abundantly in mushroom mycelium. Beta-glucans have been studied for their potential to enhance immune function and combat diseases, including cancer, infections, and autoimmune disorders.

Polysaccharide Peptides: Certain mushroom mycelium species produce polysaccharide peptides, complex molecules with potent antioxidant and anti-inflammatory properties.

Antioxidant Activity: Aromatic compounds and polysaccharide peptides found in mushroom mycelium possess significant antioxidant activity. By scavenging free radicals and reducing oxidative stress, these compounds help protect cells from damage and slow down the aging process. Antioxidant-rich mycelium extracts may therefore contribute to overall health and longevity.

Bioreactor culture mushroom mycelium represents a natural source of medicinal products, offering a rich source of bioactive compounds with diverse medicinal properties. From immune support and anti-inflammatory effects to antioxidant activity benefits, bioreactor cultures of mycelium-derived compounds hold promise for addressing a wide range of health concerns. The therapeutic potential of mushroom mycelium may lead to the development of novel treatments and supplements that promote wellness and vitality.

There are medicinal benefits of psilocybin. Psilocybin, the psychoactive compound found in certain species of mushrooms, has garnered significant attention in recent years for its potential medicinal benefits. Research suggests that when administered in controlled settings, psilocybin can offer profound therapeutic effects, particularly in treating mental health disorders such as depression, anxiety, PTSD, and addiction. The following are some of the medicinal benefits of psilocybin and its legal administration.

1. Alleviation of Depression and Anxiety: Psilocybin has shown promise in alleviating symptoms of depression and anxiety.

2. Treatment of PTSD: Post-Traumatic Stress Disorder PTSD can be debilitating, and often resistant to conventional treatments. Psilocybin-assisted therapy has emerged as a potential breakthrough in addressing PTSD.

3. Addiction Treatment: Substance use disorders pose a significant public health challenge worldwide. Psilocybin therapy has shown promise in treating forms of addiction, including alcohol, tobacco, and opioids.

4. End-of-Life Anxiety: For individuals grappling with terminal illness, existential distress, and anxiety are common. Psilocybin therapy has demonstrated efficacy in alleviating end-of-life anxiety and improving the quality of life for terminally ill patients.

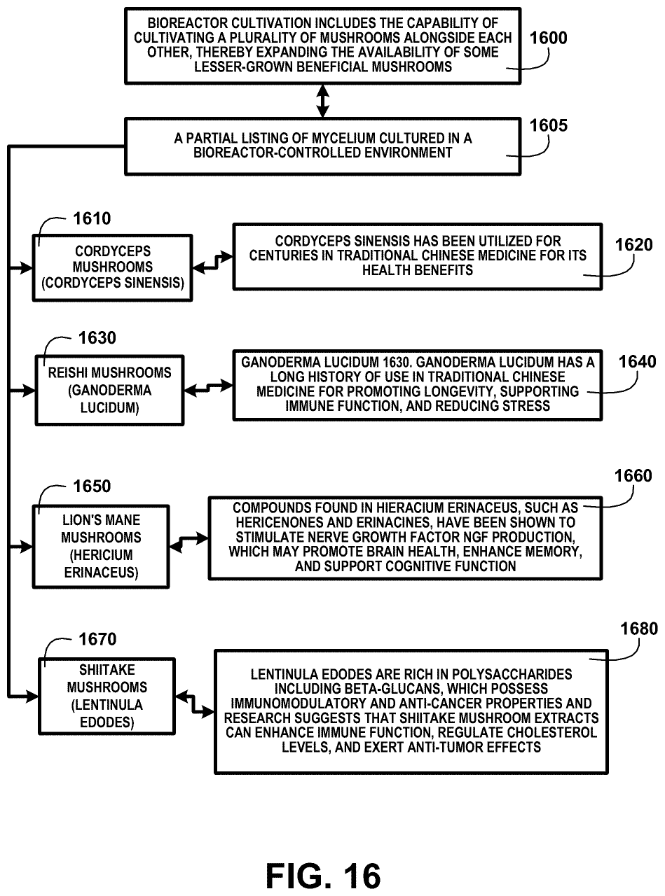

FIG. 16 shows a block diagram of an overview of types of harvested mycelium bioactive compounds of one embodiment. FIG. 16 shows bioreactor cultivation includes the capability of cultivating a plurality of mushrooms alongside each other, thereby expanding the availability of some lesser-grown beneficial mushrooms 1600. Mushrooms have long been revered in traditional medicine systems across the globe for their therapeutic properties. A partial listing of mycelium cultured in a bioreactor-controlled environment 1605 is shown as an example of the plurality of mushroom types.

One type is Cordyceps mushrooms Cordyceps sinensis 1610. Cordyceps sinensis has been utilized for centuries in traditional Chinese medicine for its health benefits 1620. Among them, Cordyceps mushrooms stand out for their remarkable health benefits and medicinal uses. In recent years, scientific research has begun to uncover the mechanisms behind these benefits, shedding light on their potential applications in modern medicine. Additionally, other mushrooms and their components have also shown promising medical properties, contributing to the growing interest in mycological medicine.

Different mushrooms contain differing components that studies have shown are more effective in use for different medical conditions. Bioreactor cultivation includes the capability of cultivating a plurality of mushrooms alongside each other, thereby expanding the availability of some lesser-grown beneficial mushrooms.

Several types of mushrooms include the Cordyceps Mushrooms. Cordyceps mushrooms, known scientifically as Cordyceps sinensis, have been utilized for centuries in traditional Chinese medicine for their purported health benefits. These fungi grow in the mountainous regions of China, Nepal, and Tibet and have been historically used to enhance vitality, improve respiratory function, and boost immune health. One of the most well-documented benefits of Cordyceps mushrooms is their immunomodulatory effects. Studies have shown that Cordyceps extracts can enhance the activity of natural killer cells, macrophages, and other immune cells, thereby strengthening the body's defense against infections and diseases.

Chronic inflammation is implicated in diseases, including cardiovascular disorders, diabetes, and cancer. Cordyceps mushrooms contain bioactive compounds such as cordycepin and polysaccharides, which exhibit potent anti-inflammatory properties. These compounds help mitigate inflammation by inhibiting pro-inflammatory cytokines and signaling pathways.

In traditional medicine, Cordyceps mushrooms are often prescribed to improve respiratory function and alleviate symptoms of respiratory disorders such as asthma and chronic obstructive pulmonary disease COPD. Research suggests that Cordyceps extracts can dilate bronchial passages, improve oxygen uptake, and reduce inflammation in the airways, thus offering relief to individuals with respiratory conditions.

Athletes and fitness enthusiasts have also turned to Cordyceps mushrooms for their ability to boost energy levels and enhance endurance. Cordyceps supplements are believed to improve Adenosine triphosphate ATP production, increase oxygen utilization, and enhance mitochondrial function, thereby promoting physical performance and stamina.

Another type is Reishi mushrooms Ganoderma lucidum 1630. Ganoderma lucidum has a long history of use in traditional Chinese medicine for promoting longevity, supporting immune function, and reducing stress 1640. Modern research has identified bioactive compounds in Reishi mushrooms, such as triterpenoids and polysaccharides, which exhibit antioxidant, anti-inflammatory, and immunomodulatory effects.

Yet another type is lion's mane mushrooms Hieracium erinaceus 1650. Compounds found in Hieracium erinaceus, such as hericenones and erinacines, have been shown to stimulate nerve growth factor NGF production, which may promote brain health, enhance memory, and support cognitive function 1660.

The last in this partial list is shiitake mushrooms lenticular edodes 1670. Lentinula edodes are rich in polysaccharides including beta-glucans, which possess immunomodulatory and anti-cancer properties and research suggests that shiitake mushroom extracts can enhance immune function, regulate cholesterol levels, and exert anti-tumor effects 1680 of one embodiment.

The invention bioreactors are versatile, scalable, and able to control parameters. These bioreactors are well-suited for use in pharmaceutical production. Bioreactors are utilized, in one embodiment, for the production of therapeutic plant-based cultivation of mushroom components for extraction in one embodiment of biopharmaceuticals.

The extraction of bioactive compounds from mushrooms from a variety of mushroom types aids in creating a sustainable supply of mushroom bioactive compounds for applications, for example, pharmaceuticals and nutraceuticals. Mushroom extracts containing polysaccharides, terpenoids, polyphenols, and other bioactive compounds are utilized in pharmaceutical formulations and nutraceutical supplements for their immunomodulatory, anti-inflammatory, antioxidant, and anticancer properties.

Other applications are in cosmetics and skincare. Mushroom-derived extracts and compounds are incorporated into cosmetics, skincare products, and cosmeceuticals for their moisturizing, anti-aging, and skin-brightening effects. Beta-glucans, polysaccharides, and phenolic compounds from mushrooms are prized for their hydrating, collagen-stimulating, and antioxidant properties.

Yet another application of the extracted mushroom bioactive compounds is in functional foods and beverages. Mushroom extracts are added to functional foods, beverages, and dietary supplements to enhance their nutritional profile and health benefits. Mushroom-derived polysaccharides, beta-glucans, and ergosterol are sought after for their cholesterol-lowering, immune-boosting, and prebiotic effects.

The extraction of bioactive compounds from mushrooms is a multifaceted process that involves a combination of methods, techniques, and applications. Extraction of the diverse chemical composition of mushrooms provides the full potential of mushroom-derived components for pharmaceutical, nutraceutical, cosmetic, and biotechnological applications.

Mushrooms, with their long history of use in traditional medicine systems, are perceived as natural and holistic remedies for health conditions. Advances in scientific research have elucidated the pharmacological properties and therapeutic potential of mushroom-derived bioactive compounds, including polysaccharides, beta-glucans, terpenoids, and polyphenols. Clinical studies and preclinical research support the efficacy of mushroom extracts in modulating immune function, reducing inflammation, and combating oxidative stress, paving the way for the development of mushroom-based medical products.

The global burden of chronic diseases such as cancer, diabetes, cardiovascular disorders, and immune-related conditions continues to rise, driving demand for complementary and alternative therapies. Mushroom-derived medical products offer potential solutions for managing symptoms, improving quality of life, and supporting conventional treatment regimens for chronic diseases.

There is a growing emphasis on preventive healthcare and wellness promotion, with consumers seeking proactive measures to maintain health and vitality. Mushroom-derived medical products, known for their immune-modulating, antioxidant, and anti-inflammatory properties, are positioned as preventive supplements for bolstering immune function, enhancing resilience, and promoting overall well-being.

The benefits of these bioreactors include increased efficiency and productivity. The bioreactors provide precise control of growth conditions, nutrient supply, and metabolic pathways, leading to enhanced yields, faster production rates, and improved process efficiency. Bioreactors facilitate quality control and process monitoring with real-time monitoring and analysis of process parameters, allowing early detection of deviations, optimization of conditions, and ensuring consistent product quality and purity. The bioreactors are highly scalable, allowing a seamless transition from laboratory-scale research to industrial-scale production. The bioreactors can be adapted to accommodate varying volumes, configurations, and process requirements, providing flexibility in process development and optimization.

The bioreactors are beneficial by providing reduced environmental impact. The bioreactors offer sustainable solutions for resource utilization, waste minimization, and eco-friendly production processes, contributing to environmental conservation and reducing the ecological footprint of industrial activities. These bioreactors advance personalized medicine solutions to address pressing global challenges in healthcare, energy, and sustainability in cultivating biological organisms.

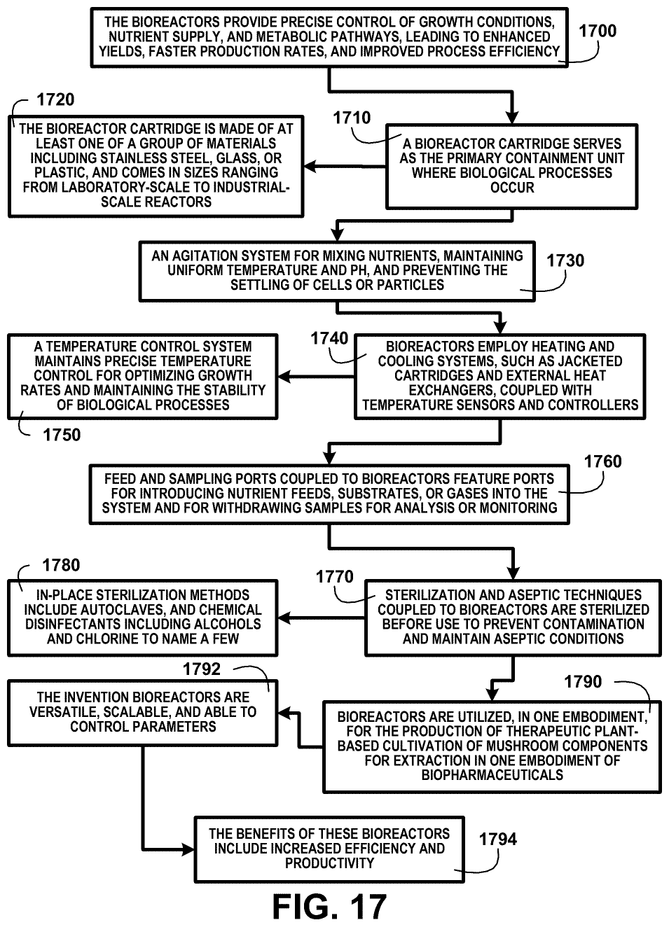

FIG. 17 shows a block diagram of an overview of bioreactor control of growth conditions of one embodiment. FIG. 17 shows the bioreactors provide precise control of growth conditions, nutrient supply, and metabolic pathways, leading to enhanced yields, faster production rates, and improved process efficiency 1700. A bioreactor cartridge serves as the primary containment unit where biological processes occur 1710. The bioreactor cartridge is made of at least one of a group of materials including stainless steel, glass, or plastic, and comes in sizes ranging from laboratory-scale to industrial-scale reactors 1720.

An agitation system for mixing nutrients, maintaining uniform temperature and pH, and preventing the settling of cells or particles 1730. Bioreactors employ heating and cooling systems, such as jacketed cartridges and external heat exchangers, coupled with temperature sensors and controllers 1740. A temperature control system maintains precise temperature control for optimizing growth rates and maintaining the stability of biological processes 1750. Feed and sampling ports coupled to bioreactors feature ports for introducing nutrient feeds, substrates, or gases into the system and for withdrawing samples for analysis or monitoring 1760.

Sterilization and aseptic techniques coupled to bioreactors are sterilized before use to prevent contamination and maintain aseptic conditions 1770. In-place sterilization methods include autoclaves, and chemical disinfectants including alcohols and chlorine to name a few 1780. Bioreactors are utilized, in one embodiment, for the production of therapeutic plant-based cultivation of mushroom components for extraction in one embodiment of biopharmaceuticals 1790. The invention bioreactors are versatile, scalable, and able to control parameters 1792. The benefits of these bioreactors include increased efficiency and productivity 1794 of one embodiment.

This invention integrates a plurality of sensors to monitor and actuate operating adjustments in, for example, temperature settings to maintain the bioreactor temperatures within predetermined ranges in one embodiment a range of 25° C. The plurality of sensors and actuators to automatically maintain operating protocols are wirelessly coupled to processors, databases, and communication devices.

The processors perform the analytics using proprietary coding and algorithms to determine mushroom culture status. Artificial intelligence and machine learning analyze photographic videos and images throughout the culturing to establish predictable growth patterns and rates of growth to compare current conditions with database-stored growth patterns and rates of growth.

The communication devices receive the sensor measurement and sensing data for transmission to the databases. The communication devices using Wi-Fi, internet, satellite, and other modes of bidirectional transmission further transmit wirelessly the processor operational monitoring results to the actuators to perform corrective action to bring the controls with the predetermined ranges of operation. The communication devices further transmit wirelessly alerts to the users to any out-of-performance protocols to initiate inspections of the equipment being monitored for any corrective maintenance or replacement of the equipment promptly to prevent damage to the culturing operations and extraction operations biomasses.

The invention includes a digital app installed on user mobile devices and a remote computer 110 of FIG. 1 to receive sensing data and status reporting. The computer 110 of FIG. 1, processors, databases, and other appropriate devices may be coupled to a remote server. The remote server may generate reports of the production, quality status, and any regulatory compliance reports for proper certifications of the bioreactor cultivation and extraction results. The periodic reporting may be prepared for submission to appropriate local, state, and federal government agencies.

The sensors include the sensing of pathogens, bacteria, and other organic constituents. Temperature, humidity, oxygenation levels, lighting levels, and other factors contribute to the detailed status of the cultivation and extraction processes. The remote server may also generate periodic maintenance reviews, predicted harvesting schedules, and other events related to the production of the bioactive compound supply status.

Process optimization is obtained by the bioreactor cultivation optimizing protocols for specific plant species and target compounds. These cultivation optimizing protocols also allow for fine-tuning growth conditions, nutrient formulations, elicitation strategies, and metabolic engineering approaches to maximize the yield, quality, and consistency of medicinal plant extracts.

The bioreactor cultivation optimizing protocols are needed for the growth patterns of mushrooms cultivated in bioreactors. Mushroom cultivation has undergone a significant transformation with the advent of bioreactors, offering precise control overgrowth conditions and enhancing productivity. Understanding the growth patterns of mushrooms within bioreactors is crucial for optimizing cultivation strategies, improving yields, and ensuring consistent quality. The growth patterns of mushrooms cultivated in bioreactors, generate the factors influencing their development, morphology, and metabolic activity.

Mushroom cultivation in bioreactors typically progresses through distinct growth phases, each characterized by specific morphological and metabolic changes. These phases include an inoculation phase that marks the introduction of mushroom spawn or mycelium into the bioreactor substrate. Mycelial growth begins as the fungal hyphae colonizes the nutrient-rich substrate, establishing a network of interconnected filaments. During the spawn run phase, mycelial growth accelerates, spreading throughout the substrate and colonizing available nutrients. The mycelium undergoes rapid expansion, forming a dense and homogeneous network of hyphae.

Primordia formation signals the initiation of fruiting body development. Triggered by environmental cues such as temperature, humidity, and light exposure, the mycelium begins to differentiate into primordial structures, known as pins or knots, at specific locations within the substrate. The fruiting phase is characterized by the emergence and maturation of mushroom fruiting bodies from the primordia. Under optimal conditions of humidity, temperature, and air exchange, the fruiting bodies undergo rapid growth, development, and morphological differentiation, culminating in mature mushrooms ready for harvest.

Several factors influence the growth patterns and productivity of mushrooms cultivated in bioreactors, including the choice of substrate and nutrient composition profoundly impact mycelial growth, fruiting body formation, and yield. Substrates rich in carbon and nitrogen sources, such as sawdust, straw, or agricultural residues, provide essential nutrients for fungal metabolism and development. Environmental factors such as temperature, humidity, light, and air exchange play a critical role in regulating mushroom growth and development. Optimal conditions promote primordia initiation, fruiting body formation, and maturation.

Agitation and mixing within the bioreactor facilitate uniform distribution of nutrients, oxygenation of the substrate, and removal of metabolic byproducts. Proper agitation ensures optimal mycelial growth and prevents substrate compaction or anaerobic conditions. pH regulation is for maintaining the physiological balance of the mushroom mycelium and optimizing enzymatic activity. Monitoring and adjusting pH levels within the bioreactor substrate help create favorable conditions for mycelial growth and fruiting body development.

Optimizing mushroom cultivation in bioreactors requires a multifaceted approach encompassing substrate formulation, environmental control, and process optimization. Optimization strategies include tailoring substrate formulations to meet the nutritional requirements of specific mushroom species and optimizing pretreatment processes to enhance substrate accessibility and digestibility for fungal colonization.

Implementing precise control and monitoring systems for temperature, humidity, light, and air exchange to create optimal conditions for mycelial growth, primordia formation, and fruiting body development. The invention provides bioreactors with appropriate agitation, mixing, aeration, and temperature control systems to promote uniform substrate colonization, prevent substrate stratification, and ensure consistent mushroom growth throughout the cultivation cycle.

Optimization includes supplementing bioreactor substrates with additional nutrients, growth promoters, or elicitors to enhance mycelial proliferation, induce primordia formation, and stimulate secondary metabolite production in mushrooms.

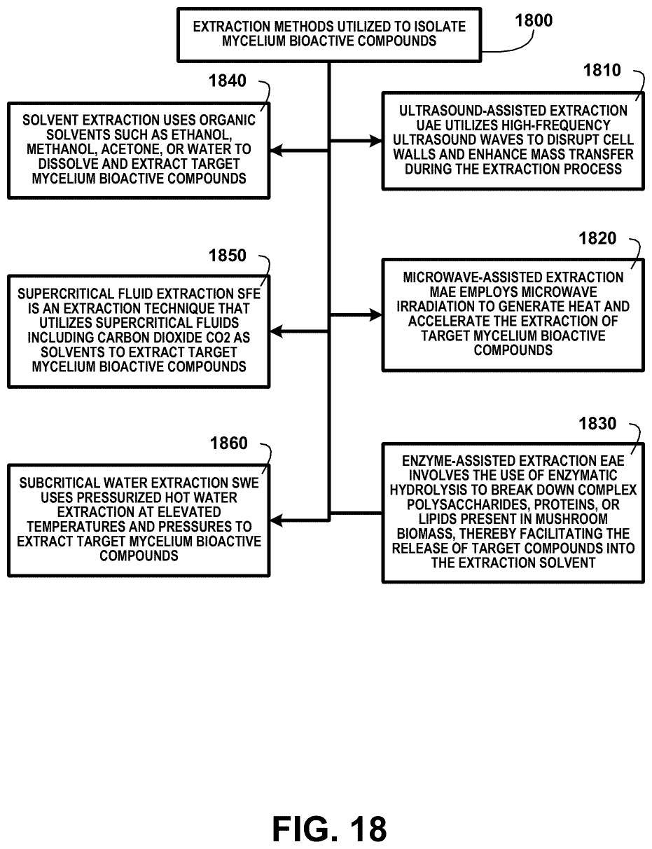

FIG. 18 shows a block diagram of an overview of the extraction of mycelium bioactive compounds of one embodiment. FIG. 18 shows extraction methods utilized to isolate mycelium bioactive compounds 1800. Ultrasound-assisted extraction UAE utilizes high-frequency ultrasound waves to disrupt cell walls and enhance mass transfer during the extraction process 1810. Microwave-assisted extraction MAE employs microwave irradiation to generate heat and accelerate the extraction of target mycelium bioactive compounds 1820. Enzyme-assisted extraction EAE involves the use of enzymatic hydrolysis to break down complex polysaccharides, proteins, or lipids present in mushroom biomass, thereby facilitating the release of target compounds into the extraction solvent 1830.

Solvent extraction uses organic solvents such as ethanol, methanol, acetone, or water to dissolve and extract target mycelium bioactive compounds 1840. Supercritical fluid extraction SFE is an extraction technique that utilizes supercritical fluids including carbon dioxide CO₂ as solvents to extract target mycelium bioactive compounds 1850. Subcritical water extraction SWE uses pressurized hot water extraction at elevated temperatures and pressures to extract target mycelium bioactive compounds 1860 of one embodiment.

Another embodiment of extraction is subcritical water extraction SWE. SWE, also known as pressurized hot water extraction or hydrothermal extraction, utilizes water at elevated temperatures and pressures to extract target compounds from mushroom biomass. SWE is effective for extracting polar and semi-polar compounds, such as polysaccharides and phenolic compounds, while minimizing the need for organic solvents and preserving the native structure and bioactivity of the extracted components. In addition to extraction methods, techniques are employed to enhance the efficiency, yield, and specificity of mushroom component extraction. Embodiments of extraction also include ultrasound-assisted extraction UAE. UAE utilizes high-frequency ultrasound waves to disrupt cell walls and enhance mass transfer during the extraction process. Ultrasonic cavitation promotes the release of intracellular components from mushroom biomass, resulting in higher extraction yields and reduced extraction times. UAE is particularly effective for extracting polysaccharides, terpenoids, and phenolic compounds from mushrooms, offering advantages such as shorter extraction times, lower solvent consumption, and improved extraction efficiency.

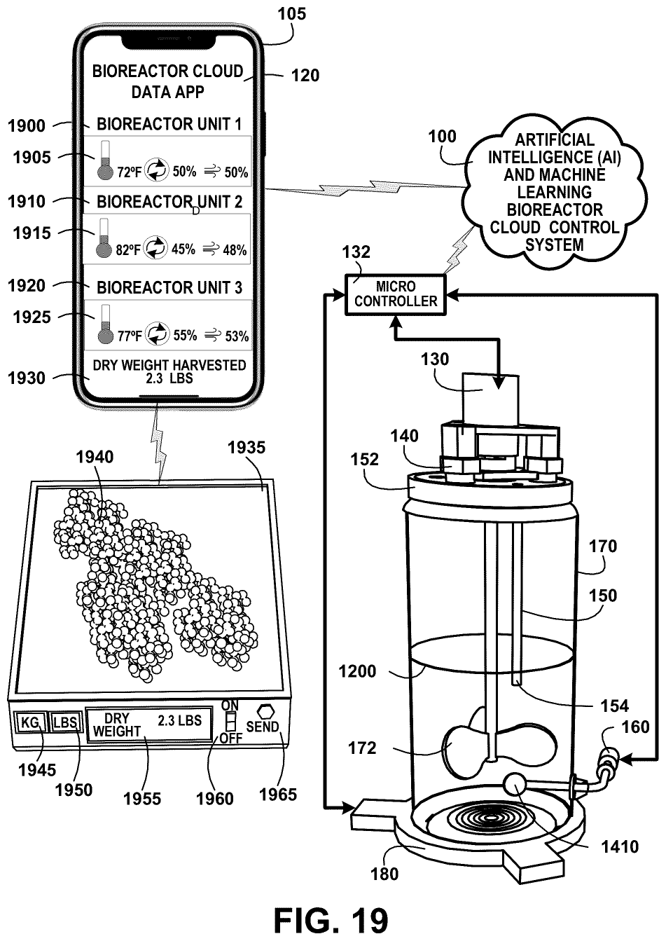

FIG. 19 shows for illustrative purposes only an example of the bioreactor cloud data app of one embodiment. FIG. 19 shows the artificial intelligence (AI) and machine learning bioreactor cloud control system 100 wirelessly coupled to the microcontroller 132 having the bioreactor cloud data app 120 and a user's mobile device 105 having a bioreactor cloud data app 120. The microcontroller 132 coupled to the artificial intelligence (AI) and machine learning bioreactor cloud control system 100 regulates the operations of at least a motor driver 130 coupled to magnetic motor support 140 of the lid 152, the agitator 172 coupled to the motor driver 130, the heating element base 180, the thermistor 154 inserted into the thermistor tube 150 to measure the temperature of the substrate, and the oxygenation air pump 160 of FIG. 1 supplying airflow to the airstone 1410 of FIG. 14. The substrate level 1200 is shown in the bioreactor cartridge 170.

The user's mobile device 105 bioreactor cloud data app 120 displays the bioreactor units the user has registered, each with a unique serial number. Displayed is bioreactor unit 1-1900 showing unit 1 settings temp 72° F., rpm 50%, and oxygenation 50% 1905. Also shown are bioreactor unit 2 1910-unit 2 settings temp 82° F., rpm 45%, and oxygenation 48% 1915, and bioreactor unit 3 1920-unit 3 settings temp 77° F., rpm 55%, and oxygenation 53% 1925.

The user placed the dried harvested mycelium on a user digital scale 1935 and transmitted the dry weight harvested 2.3 lbs. 1930 feedback to the bioreactor cloud data app 120. The feedback to the bioreactor cloud data app 120 transmits the user feedback to the artificial intelligence (AI) and machine learning bioreactor cloud control system 100. The harvested weight is analyzed by artificial intelligence (AI) and machine learning bioreactor cloud control system 100 with comparisons of other harvested outcomes and the operating settings during cultivation to determine settings that produce the optimum harvests. This analysis is used in making recommendations on settings to allow users to optimize the cultivation process.

The user digital scale 1935 weighs the dried harvested mycelium 1940 placed on the scale in kg 1945 or lbs. 1950 selected by the user. The user digital scale 1935 includes an on/off switch 1960 and displays the dry weight of 2.3 lbs. 1955 which the user can select to send 1965 of one embodiment.

The bioreactor system cultivates the mycelium root structures into a natural product. This product has an exact, consistent potency containing all the natural compounds found in these fungi. Typically, it takes 6-8 weeks to fully grow mushrooms, and using the bioreactor system the production of the active ingredients is only 7-10 days.

Traditional mushroom cultivation requires extensive infrastructure and costly lab equipment, posing significant barriers to small-scale producers. The bioreactor system reduces costs, simplifies the process, and ensures safe, high-quality production, making it accessible to a broader audience.

Naturally occurring bioactive compounds found in certain mushrooms have shown significant promise in clinical studies for treating mental health conditions such as depression, anxiety, PTSD, and addiction. Growing mycelium is an integral part of mushroom cultivation. It is most often grown by cloning it from one growth medium to another. Mushroom mycelium can also be grown from spores.

Mushroom-producing fungi are, by volume and mass, mostly composed of a filamentous mycelium. Mycelium looks like a web-like branching network of white fungal strands. Mycelium in nature can be found under leaf litter, rotting logs, and even sometimes in the soil. Only once matured will this mycelium begin producing mushrooms. Mycelium grows within substrates that act as their home and food source. Substrates are the natural materials that fungi inhabit. Common substrates are wood, straw, grain, cardboard, and organic waste. Mycelium grows from spores that are the reproductive cells of the mushroom. For cultivation, the spores are germinated. Cultivation of mycelium via spores is an advanced technique. This usually requires lab equipment and skill. For beginners, it is much better to start with a clean and healthy mycelium culture.

Mushroom spawn is mycelium, usually grown on grain and used for inoculation. Inoculation is the process of introducing mycelium into a new growth medium. High-quality mushroom spawns should grow vigorously and be free of any contaminants. Cultivation equipment is well-cleaned and rubbed down with 70% alcohol. Pasteurization is a process in which you remove antagonistic microorganisms from your substrate. There are many ways to pasteurize your substrate. The easiest is to submerge your substrate in water for 1 hour at 160° F. Alternatively, submerge your substrate in 0.2% activated lime water for 12 hours. After pasteurization, place your substrate to drain and cool down. If producing biomaterials, inoculate your mushrooms within a specialized mold. Make sure it has air exchange and proper growing conditions.

Incubate growing mushroom mycelium in liquid culture. Incubation promotes Mycelium growth in a liquid medium. The liquid medium in the nutrient container is supplemented with nutrients to provide proper growth ingredients to produce the best growth of the Mycelium. The incubator regulates an optimum temperature to stimulate the growth of the spores. If the temperature is too low, the growth rate slows down. Low temperatures can lead to slower colonization of the substrate and reduced biomass accumulation The growth ingredients are added to a sugar-water solution inside a nutrient container with a lid. The liquid solution within the nutrient container provides the proper mixture of nutrients for the specific Mycelium spore species. The lid of the nutrient container has a “self-healing injection port” to inoculate the liquid culture with the best growth spores into new growth mediums with a syringe.

Temperature control modulators are configured to maintain the temperature settings stored in the bioreactor cloud platform. The temperature settings are based on the best growth results for each mycelium spore strain. The bioreactor cloud platform will automatically enter the temperature settings into the nutrient container and bioreactors upon the start of a growth cycle. Optimum proper growth conditions produce higher yields of the best growth spores and increase harvests of the mycelium products.

Temperature plays a role in the cultivation of mycelium in a bioreactor. Mycelium has an optimal temperature range for growth. For many species, this range is between 20° C. and 30° C. 68° F. to 86° F., though it can vary depending on the specific fungal strain. Within this range, the mycelium grows most efficiently, with optimal rates of nutrient uptake and metabolic activity. In a temperature within the optimal range, the mycelium grows rapidly. However, if the temperature is too low, the growth rate slows down. Low temperatures can lead to slower colonization of the substrate and reduced biomass accumulation.

Elevated temperatures can increase metabolic activity but can also stress the mycelium. If temperatures exceed the optimal range, it can lead to reduced growth rates, nutrient depletion, and even death of the mycelium. Prolonged exposure to elevated temperatures may also increase the risk of contamination. Low temperatures can slow down or halt growth. Prolonged exposure to low temperatures may lead to poor colonization and reduced yields. In some cases, extremely low temperatures might even cause damage to the mycelial structure.

Temperature affects not only growth but also the productivity and yield of the mycelium. Maintaining an optimal temperature can lead to higher yields and better quality of the fungal product. For example, in industrial applications, temperature control is crucial for maximizing mushroom production or the yield of mycelium-based products. Temperature influences metabolic processes within the mycelium, including enzyme activity and nutrient metabolism. Proper temperature regulation ensures that these processes occur efficiently, supporting healthy mycelial growth and development. Temperature also plays a role in controlling microbial contamination. Maintaining a stable and appropriate temperature can help minimize the risk of contamination by unwanted microorganisms. Controlling temperature in a bioreactor is for optimizing mycelium cultivation. Maintaining temperatures within the species-specific optimal range ensures efficient growth, productivity, and quality of the mycelial biomass.

Agitation of the growing medium in mycelium cultivation, in a bioreactor with a liquid culture system, plays a role in improving the growth and development of spores. Agitation helps to ensure an even distribution of oxygen throughout the growing medium. Mycelium, the vegetative part of fungi, requires a constant supply of oxygen to support aerobic respiration and overall growth. Agitation prevents oxygen depletion and promotes efficient gas exchange. Agitation helps to evenly distribute nutrients throughout the medium. This ensures that all parts of the growing medium receive an adequate supply of nutrients, for consistent and healthy mycelial growth.

Agitation can aid in maintaining uniform temperature distribution within the bioreactor. It helps to prevent localized temperature gradients that could negatively impact mycelial growth and development. In liquid cultures, agitation prevents the settling of mycelium or spores at the bottom of the container. This is particularly important in liquid fermentation systems where uniform growth throughout the medium is desired. Agitation ensures a homogeneous suspension of spores or mycelium in the medium. This is important for maintaining consistency in growth rates and ensuring that the mycelium colonizes the medium evenly.

By improving oxygen and nutrient availability, agitation can lead to faster growth rates of the mycelium. This can be beneficial in both research and industrial applications where rapid mycelial expansion is desired. Agitation can help to reduce the risk of contamination by preventing the establishment of localized conditions where contaminants might thrive. However, it is important to balance agitation with other factors to minimize the risk of mechanical damage to the mycelium or excessive shear forces that might stress it. Overall, agitation is a factor in optimizing the growth environment for mushroom spores in liquid cultures or bioreactors. It helps to create a more uniform and favorable growth environment, supporting the efficient and healthy development of the mycelium.

Oxygenation is used in cultivating mycelium in a bioreactor. Mycelium, being an aerobic organism, relies on oxygen for physiological and metabolic processes. Mycelium primarily uses aerobic respiration to generate energy. Oxygen is required for the complete breakdown of substrates, leading to the production of ATP adenosine triphosphate, which is essential for growth and metabolism. Inadequate oxygenation can lead to a shift from aerobic to anaerobic respiration, which is less efficient and can result in the accumulation of toxic by-products.

Adequate oxygen levels support optimal growth rates and biomass production. In a bioreactor, sufficient oxygenation ensures that the mycelium grows efficiently, resulting in higher yields and better quality of the fungal product. Oxygenation affects nutrient uptake and utilization. Proper oxygen levels enhance the efficiency of nutrient metabolism, which is crucial for maintaining healthy mycelial growth and development.

In large-scale or high-density cultures, oxygen demand can exceed the supply if not effectively managed. Oxygen limitation can lead to reduced growth rates, slower colonization, and poor overall performance. Effective aeration strategies are needed to ensure that oxygen is adequately supplied throughout the culture. Inadequate oxygenation can lead to the production of unwanted metabolic by-products, such as ethanol and other fermentation products. These by-products can inhibit growth and affect the overall health of the mycelium.

In a bioreactor, effective oxygenation helps in maintaining a uniform environment. This includes the even distribution of oxygen throughout the culture medium, preventing localized areas of low oxygen that can negatively impact growth. The design of the bioreactor, including agitation and aeration systems, is often tailored to meet the oxygen demands of the mycelium. Properly designed bioreactors ensure that oxygen is efficiently supplied and distributed, supporting optimal growth conditions.

In summary, oxygenation increases the successful cultivation of mycelium in a bioreactor. It directly impacts growth rates, productivity, nutrient utilization, and overall health of the mycelium. Effective oxygen management strategies are essential to maintain a favorable growth environment and achieve desired outcomes in mycelium cultivation.

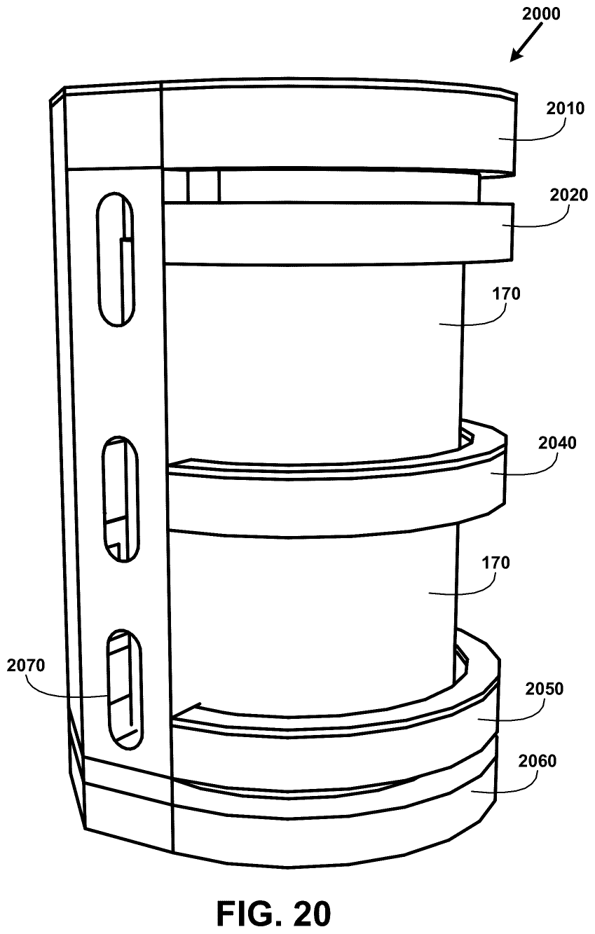

FIG. 20 shows for illustrative purposes only an example of a magnetic collar of one embodiment. FIG. 20 shows a bioreactor 2000 having a plurality of a magnetic collar 2010. The bioreactor cartridge 170 is held tightly in place with the plurality of a magnetic collar 2010 including magnetic collar 2010, magnetic collar 2020, magnetic collar 2040, magnetic collar 2050, and magnetic collar 2060. The bioreactor 2000 includes ventilation openings 2070 to provide airflow to control the temperature within the bioreactor of one embodiment.

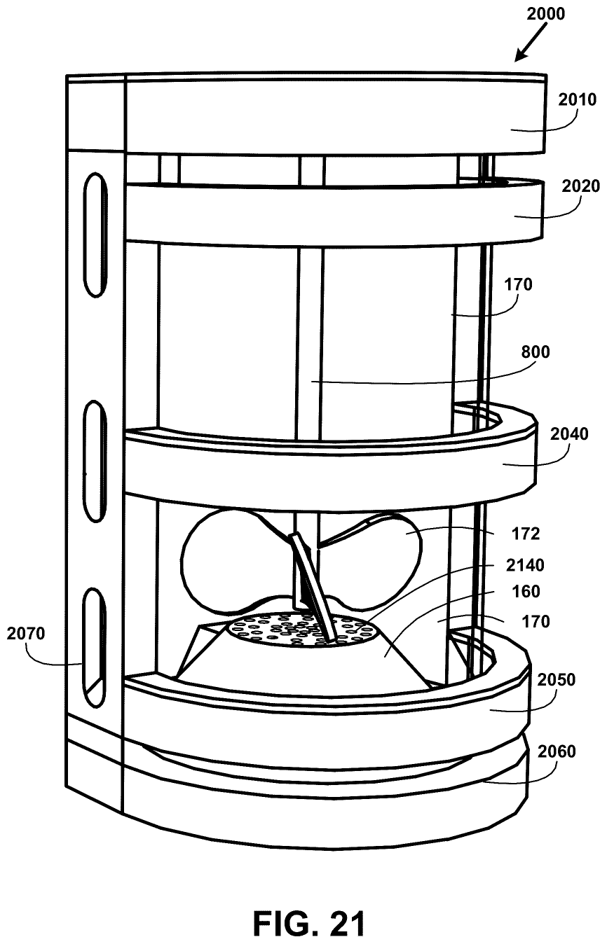

FIG. 21 shows for illustrative purposes only an example of an agitator rotor rod of one embodiment. FIG. 21 shows the bioreactor 2000 having magnetic collar 2010, magnetic collar 2020, magnetic collar 2040, magnetic collar 2050, and magnetic collar 2060 used to secure tightly the bioreactor cartridge 170. The bioreactor 2000 includes ventilation openings 2070 to provide airflow to control the temperature within the bioreactor. Also showing is the agitator rotor rod 800 coupled to the agitator 172. The oxygenation air pump 160 pumps oxygen through cartridge oxygenation airflow inlets 2140 to provide oxygen to the sterile substrate to promote the growth of the mushroom spores of one embodiment.

The bioreactor cartridge contains a sterile substrate into which mycelium spores are injected prior to placing into the bioreactor. The bioreactor includes a front pivoting section of the top section to open for the installation of a bioreactor cartridge. When placed in the bioreactor the motor is connected to the cartridge agitator rod. Upon closing the pivoting section magnetic collars are coupled to the bioreactor to hold the cartridge securely in place. Gears are engaged to operate the agitator motor. The control display screen is activated. The heating element is located at the bottom of the bioreactor to heat the sterile substrate and spores. The air filter and oxygenation aerator motor are connected to the microcontroller and air stone tube to begin injecting oxygen into the sterile substrate and spores. The bioreactor cartridge begins operations for rotating the agitator, oxygenation aerator motor, and heating the sterile substrate and spores.

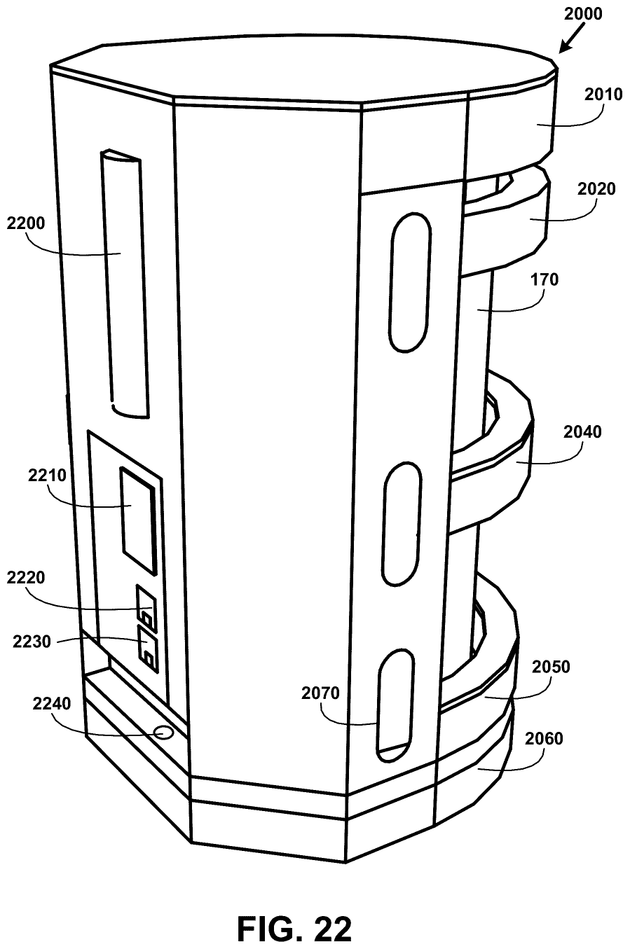

FIG. 22 shows for illustrative purposes only an example of a bioreactor cartridge control of one embodiment. FIG. 22 shows the bioreactor 2000 having the magnetic collar 2010, magnetic collar 2020, magnetic collar 2040, magnetic collar 2050, and magnetic collar 2060 used to secure the bioreactor cartridge 170. The bioreactor 2000 includes ventilation openings 2070 to provide airflow to control the temperature within the bioreactor. Also showing is one embodiment of a bioreactor cartridge thermistor tube 2200 for insertion of a thermistor 154 of FIG. 1. FIG. 22 also shows a control display screen 2210 to display the temperature, oxygen level, and rpm of the agitator to allow the user to monitor the cultivation progress.

Also shown is an oxygen saturation gauge 2220 to display the saturation level of oxygen in the substrate for adjusting the flow rate of the oxygenation air pump 160 of FIG. 1. A nutrient sensor gauge 2230 displays sensor readings of nutrient levels in the substrate to determine supplemental nutrient amounts to be added to the substrate. A reset power button 2240 allows the user to reset the gauges to get accurate current readings.

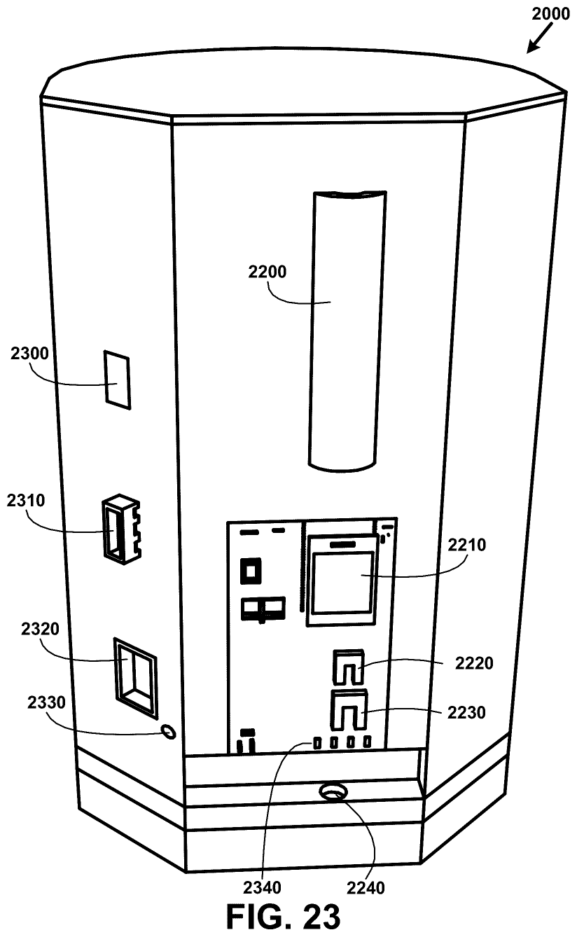

FIG. 23 shows for illustrative purposes only an example of the control display screen of one embodiment. FIG. 23 shows the bioreactor cartridge thermistor tube 2200 and the control display screen 2210. Also seen are the oxygen saturation gauge 2220, nutrient sensor gauge 2230, and reset power button 2240. Also showing are LED status lights 2340. A power light 2300 is lit when power is connected to the bioreactor 2000 and not lit when power is off. A USB connector 2310 allows the user to connect USB devices to, for example, record current readings for historical progress of the cultivation. A substrate sample outlet 2320 allows the user to get a test sample of the substrate for testing cultivation conditions that affect the growth of the mushroom spores 176 of FIG. 1. A sample button 2330 is used to turn on a suction pump to extract the substrate sample of one embodiment.

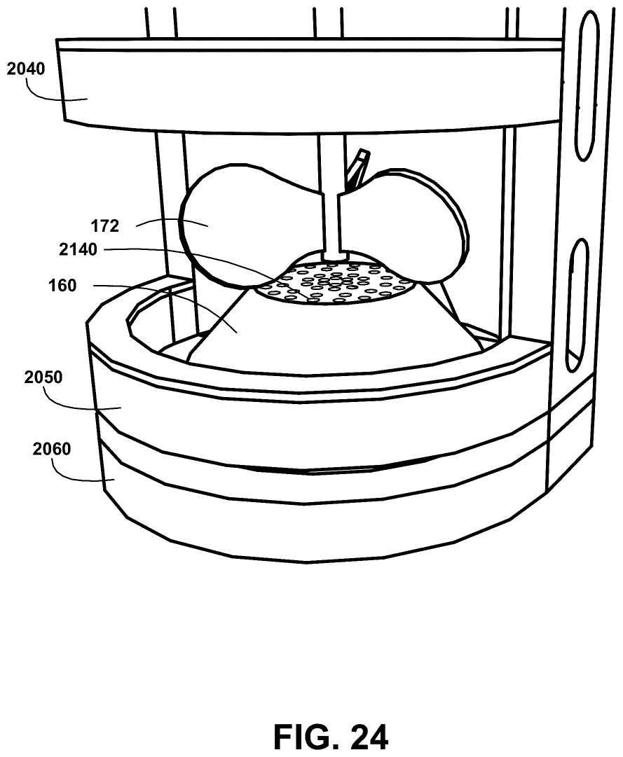

FIG. 24 shows for illustrative purposes only an example of oxygenation air flow vents of one embodiment. FIG. 24 shows the magnetic collar 2040, the magnetic collar 2050, and the magnetic collar 2060. Also showing is the agitator 172, oxygenation air pump 160, and oxygenation air flow vents 2140.

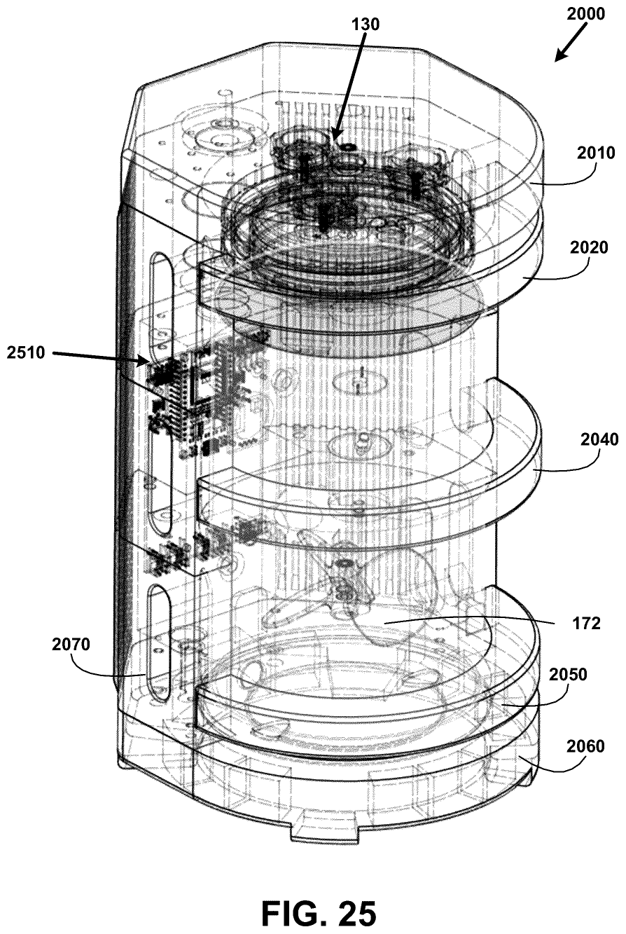

FIG. 25 shows for illustrative purposes only an example of a transparent view of the bioreactor of one embodiment. FIG. 25 shows a transparent view of the bioreactor 2000 with the bioreactor cartridge secured with the magnetic collar 2010, magnetic collar 2020, magnetic collar 2040, magnetic collar 2050, and magnetic collar 2060.

The ventilation openings 2070 provide airflow to control the temperature within the bioreactor 2000. The control panel 2510 is seen from the back inside view and includes the control components. The motor driver 130 is seen on top and is used to rotate the agitator 172 at predetermined rpm settings that are adjusted using artificial intelligence to regulate the settings of one embodiment.

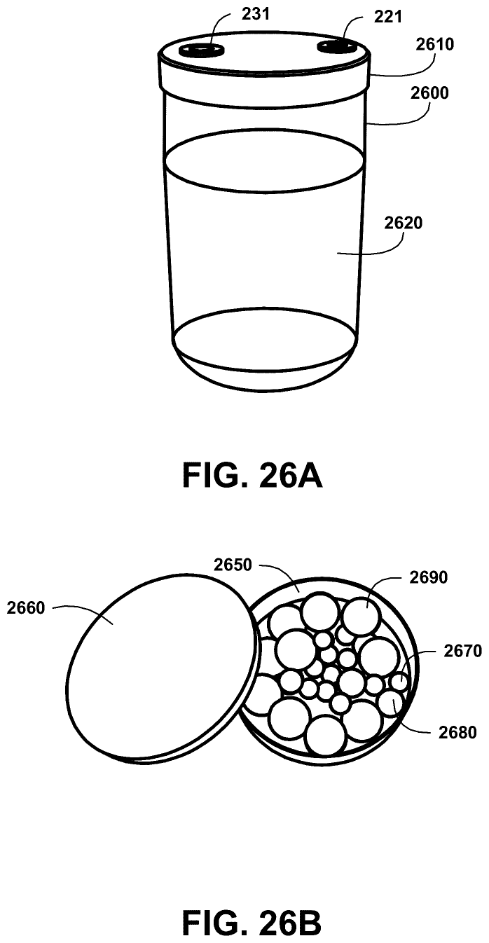

FIG. 26A shows for illustrative purposes only an example of mycelium liquid culture nutrients in a liquid solution of one embodiment. FIG. 26A shows a nutrient liquid container 2600 with a cover 2610. Inside the container is a sterile liquid substrate 2620, to which nutrients are added through a dry nutrient fill port 221. This mixture provides essential growth ingredients for cultivating spores. Once prepared, the spores are injected into the nutrient solution, and the best growth spores identified based on growth are added to foster growth.

The culture continues to expand in the liquid, ensuring an adequate supply of spores for cartridge distribution. A cartridge is the growth module that is coupled to the bioreactor to monitor the growth of the mycelium inoculated into the nutrient solution held in the cartridge. The bioreactor receives settings for the control modules of the bioreactor that are coupled to the agitator, oxygenation aerator motor, and heating element of the cartridge. The agitator rpm is adjusted during the growth cycle to prevent the spores from clumping together as more growth occurs and provides spacing between spores for access to oxygen and nutrients. The oxygenation aerator motor passes air through an air filter to provide oxygen to the spores to maintain the growth of the spores. The heating element is regulated to provide heat within the optimum range of the spore strain. The settings are adjusted by the cloud platform as the growth cycle progresses. The cartridge includes an injection port to add supplemental nutrients to keep the nutrient levels at a level to maintain adequate supplies to the spores as the numbers increase with the growth.

The cartridges are sent to users with the sterile liquid substrate within the cartridge. The cultured spores are sent separately in a syringe to inject into the cartridge to begin the growth cycle.

The supply of spores for different strains is initially cultivated using an incubator to regulate the temperature with the optimum range of temperatures for each strain held in a dish. After 7 to 14 days the spore growth is evaluated and the best-growth spores are segregated from poorer-growth spores for inoculation into a nutrient container. The nutrient container holds a liquid solution to continue the growth of the best-growth spores. The growth of the spores in the nutrient container builds a larger supply of the best-growth spores to extract with a syringe to feed the spores into the cartridge.

A liquid nutrient extraction port 231 allows sterile extraction of the nutrient solution, which can be transferred into cartridges without living organisms, awaiting spore inoculation. The nutrient solution contains components tailored to support mycelium strains.

FIG. 26B shows for illustrative purposes only an example of plating spores to the culture of one embodiment. FIG. 26B shows a mycelium culture dish 2650 and a dish cover 2660. Spores are placed in the dish with nutrients and cultured for 7 to 14 days. Afterward, the spores are genetically analyzed to classify them into three categories: poor growth spores 2670, medium growth spores 2680, and best growth spores 2690. The best growth spores 2690 are selected, and poor or medium spores are removed. The best spores continue to grow and multiply, in one embodiment moved to larger cartridges to ensure an adequate quantity. This process ensures a strong culture for inoculation into nutrient solutions.

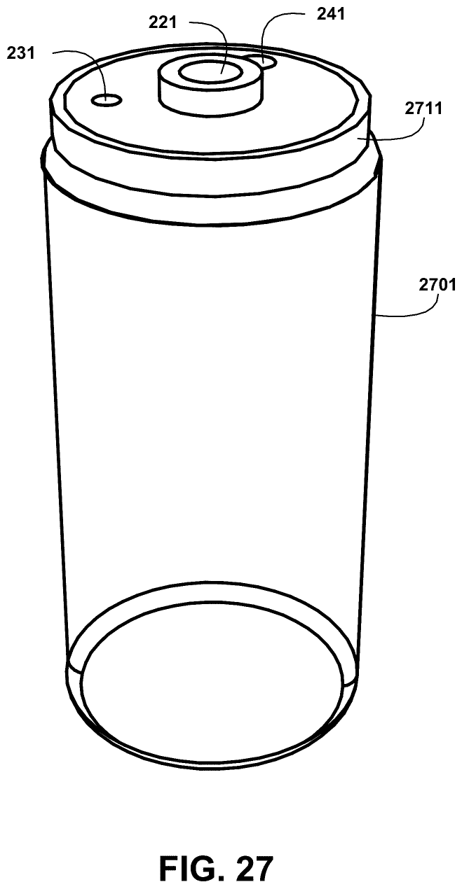

FIG. 27 shows for illustrative purposes only an example of the refillable growing medium cartridge of one embodiment. FIG. 27 shows a refillable growing medium cartridge 2701 with a cover 2711 that contains an injection sterile port 221, a nutrient injection port 231, and a pressure relief valve 241. The pressure relief valve 241 helps manage excess pressure created when heating spores in the nutrient solution. The sterile port allows for spore injection into a bioreactor container cartridge referred to as part A, where spores are introduced using a 10cc syringe after the nutrient solution has been added. The cartridge part B contains liquid nutrients such as dextrose, sugars, and nitrogen that support mycelium growth. The best growth spores are injected into this nutrient-rich solution.

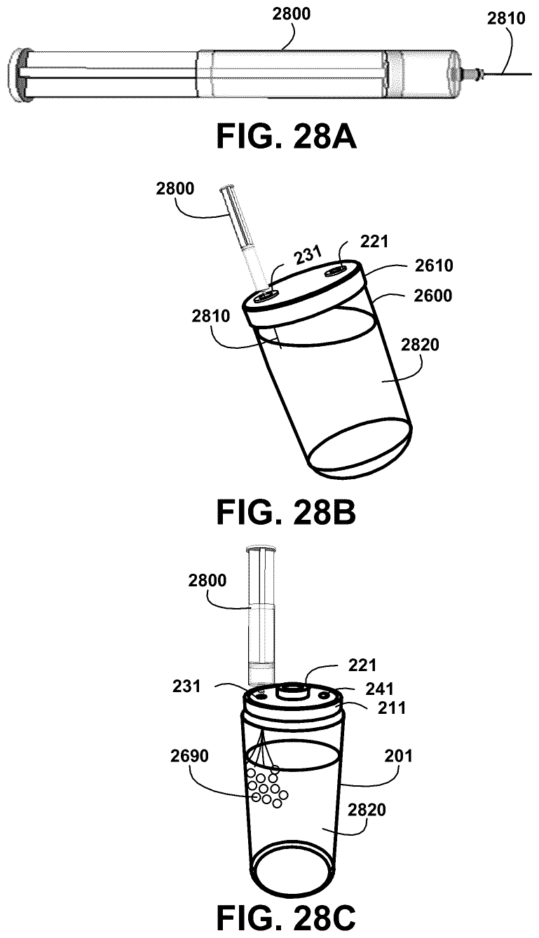

FIG. 28A shows for illustrative purposes only an example of a syringe of one embodiment. FIG. 28A shows illustrates a 10cc syringe 2800 with an attached needle 2810. The syringe is filled using a sterile process and shipped in sterile packaging. It is used for injecting the best growth spores 2790 of FIG. 27B into the liquid growth nutrients 2820 of FIG. 28B in the cartridge, maintaining a sterile environment.

FIG. 28B shows for illustrative purposes only an example of a syringe extracting liquid nutrients from one embodiment. FIG. 28B shows the syringe 2800 and needle 2810 extracting liquid growth nutrients 2820 through the extraction port 231 on the cover 2610, which are mixed with cultured best growth spores 2790 of FIG. 27B from the nutrient liquid container 2600. Nutrients are added through the dry nutrient fill port 221, and the liquid nutrient extraction port 231 is used to extract nutrient solution without living organisms, or with best growth spores, depending on the process.

FIG. 28C shows for illustrative purposes only an example of a syringe injecting the best growth spores of one embodiment. FIG. 28C shows the syringe 2800 injecting best growth spores 2690 into the liquid growth nutrients 2820 contained in the bioreactor cartridge 201. The container holds a liquid substrate with liquid growth nutrients 2820. Cover 211 of the container features both the nutrient injection port 231 and the sterile port 221 for injecting the nutrients and spores.

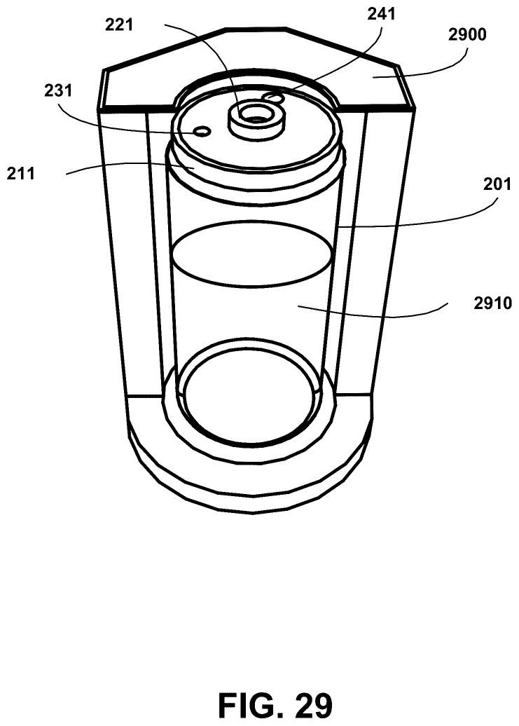

FIG. 29 shows for illustrative purposes only an example of a refillable growing medium cartridge placed in a bioreactor of one embodiment. FIG. 29 shows the refillable growing medium cartridge 201 placed inside a bioreactor 2900. The cartridge cover 211 includes a sterile port 221 for spore injection into the liquid nutrient 2910. The nutrient injection port 231 is used to add additional nutrients as needed during the cultivation process. Also present is a pressure relief valve 241 to prevent pressure build-up.

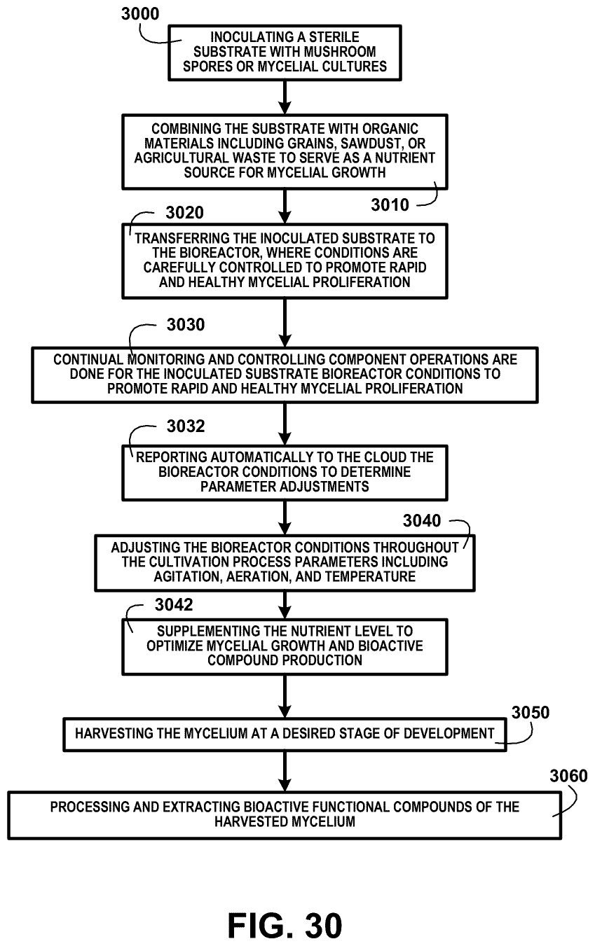

FIG. 30 shows a block diagram of an overview flow chart of the bioreactor cultivation of mushrooms of one embodiment. FIG. 30 shows inoculating a sterile substrate with mushroom spores or mycelial cultures 3000. The mushroom spores are fed nutrients by combining the substrate with organic materials including grains, sawdust, or agricultural waste to serve as a nutrient source for mycelial growth 3010. Reaching a predetermined growth the process continues by transferring the inoculated substrate to the bioreactor, where conditions are carefully controlled to promote rapid and healthy mycelial proliferation 3020. Continual monitoring and controlling component operations are done for the inoculated substrate bioreactor conditions to promote rapid and healthy mycelial proliferation 3030.

The control includes reporting automatically to the cloud the bioreactor conditions to determine parameter adjustments 3032. Adjusting the bioreactor conditions throughout the cultivation process parameters including agitation, aeration, and temperature 3040 improves cultivation results. The spores grow well by supplementing the nutrient level to optimize mycelial growth and bioactive compound production 3042. The controlled bioreactor cultivation results in harvesting the mycelium at a desired stage of development 3050. Harvesting is followed by processing and extracting bioactive functional compounds of the harvested mycelium 3060 of one embodiment.

The bioactive compounds extracted from the bioreactor cultured mycelium include Polysaccharides. Mushroom mycelium cultivated in bioreactors is rich in polysaccharides, and complex carbohydrates with diverse physiological effects. Beta-glucans, a type of polysaccharide found in many mushroom species, exhibit immunomodulatory properties and have been studied for their potential to boost immune function and combat diseases.

Cultivating mushroom mycelium in the bioreactors represents an innovative approach to harnessing nature's pharmacy and unlocking the therapeutic potential of bioactive compounds. By optimizing conditions for mycelial growth and extraction the production of high-quality extracts rich in polysaccharides, triterpenoids, and other valuable compounds is increased in volume and quality. The optimization includes proprietary formulation of bioreactor substrates including nutrients formulated to promote the cultured mushroom mycelium growth and quality. Additionally, optimization includes proprietary formulation of extraction liquids to increase the quantity and quality of the extracted bioactive compounds.

These bioactive compounds offer a wide range of medicinal benefits, from immune modulation and anti-inflammatory effects to antioxidant activity. The bioreactor technology of this invention improves the development of novel therapeutics and nutraceuticals derived from cultured mushroom mycelium.

Mushroom Mycelium is cultured in the bioreactors as a source of medicinal bioactive compounds. Mushroom mycelium, the intricate network of thread-like structures that form the vegetative part of fungi, holds remarkable potential as a source of bioactive compounds with medicinal benefits. Mycelium, often referred to as the “hidden half” of fungi, plays a crucial role in nutrient cycling, soil health, and ecosystem resilience. Beyond its ecological significance, mycelium harbors a treasure trove of bioactive molecules that have been studied for their therapeutic properties.

Bioreactor culture mushroom mycelium represents a natural source of medicinal products, offering a rich source of bioactive compounds with diverse medicinal properties. From immune support and anti-inflammatory effects to antioxidant activity benefits, bioreactor cultures of mycelium-derived compounds hold promise for addressing a wide range of health concerns. The therapeutic potential of mushroom mycelium may lead to the development of novel treatments and supplements that promote wellness and vitality.

Mycelium, as the vegetative part of fungi, is indeed a versatile and functional organism with numerous applications, particularly in the realm of health and wellness. Medicinal Properties: Mycelium produces a wide array of bioactive compounds, including polysaccharides, terpenoids, and phenolic compounds, which have been studied for their medicinal properties. These compounds exhibit biological activities such as anti-inflammatory, antioxidant, immunomodulatory, and antimicrobial effects.

Nutritional Benefits: Some species of mushrooms grown from mycelium are not only delicious but also highly nutritious. They are rich sources of protein, dietary fiber, vitamins (such as B vitamins and vitamin D), and minerals (such as selenium and potassium). Incorporating these mushrooms into diets can help improve overall nutrition and contribute to better health outcomes. Gut Health: Mycelium-derived products, particularly those containing prebiotic fibers, can support gut health by promoting the growth of beneficial gut bacteria. These fibers serve as food for probiotics, helping to maintain a healthy balance of microorganisms in the gut microbiome. A balanced gut microbiome is associated with improved digestion, enhanced immune function, and reduced risk of gastrointestinal disorders.

Mushroom Mycelium is cultured in the bioreactors as a source of medicinal bioactive compounds. Beyond its ecological significance, mycelium harbors bioactive molecules that have been studied for their therapeutic properties and the potential medicinal benefits they offer.

Bioactive Compounds Found in Mushroom Mycelium include Beta-Glucans: Beta-glucans are polysaccharides found abundantly in mushroom mycelium. These compounds possess immunomodulatory properties, stimulating the activity of macrophages, natural killer cells, and other components of the immune system. Beta-glucans have been studied for their potential in enhancing immune function and combating diseases, including cancer, infections, and autoimmune disorders.

Polysaccharide Peptides: Certain mushroom mycelium species produce polysaccharide peptides, complex molecules with potent antioxidant and anti-inflammatory properties. These compounds have shown promise in protecting cells from oxidative damage, reducing inflammation, and supporting overall health and well-being.

Triterpenoids: Triterpenoids are another class of bioactive compounds found in mushroom mycelium. These molecules exhibit diverse pharmacological activities, including anti-inflammatory, antimicrobial, and anti-cancer properties. Triterpenoids have been studied for their potential to manage chronic inflammatory conditions, prevent infections, and inhibit tumor growth.

Aromatic Compounds: Mushroom mycelium is rich in aromatic compounds, such as phenols and flavonoids, which contribute to its distinctive aroma and flavor. These compounds possess antioxidant properties and may help protect cells from oxidative stress and DNA damage. Additionally, some aromatic compounds found in mycelium have demonstrated antimicrobial activity against pathogens.

Medicinal benefits of mycelium-derived bioactive compounds include immune support with Beta-glucans and other immunomodulatory compounds found in mushroom mycelium have been shown to enhance immune function. By activating immune cells and promoting cytokine production, these compounds help the body mount a robust immune response against infections, tumors, and other threats.

Anti-Inflammatory Effects: Polysaccharide peptides and triterpenoids present in mushroom mycelium exhibit potent anti-inflammatory properties. These compounds help mitigate inflammation by suppressing the production of pro-inflammatory cytokines and inhibiting inflammatory pathways. As a result, mycelium-derived bioactive compounds may benefit individuals suffering from chronic inflammatory conditions such as arthritis, inflammatory bowel disease, and asthma.

Antioxidant Activity: Aromatic compounds and polysaccharide peptides found in mushroom mycelium possess significant antioxidant activity. By scavenging free radicals and reducing oxidative stress, these compounds help protect cells from damage and slow down the aging process. Antioxidant-rich mycelium extracts may therefore contribute to overall health and longevity.

Bioreactor culture mushroom mycelium represents a natural source of medicinal products, offering a rich source of bioactive compounds with diverse medicinal properties. From immune support and anti-inflammatory effects to antioxidant activity benefits, bioreactor cultures of mycelium-derived compounds hold promise for addressing a wide range of health concerns. The therapeutic potential of mushroom mycelium may lead to the development of novel treatments and supplements that promote wellness and vitality.

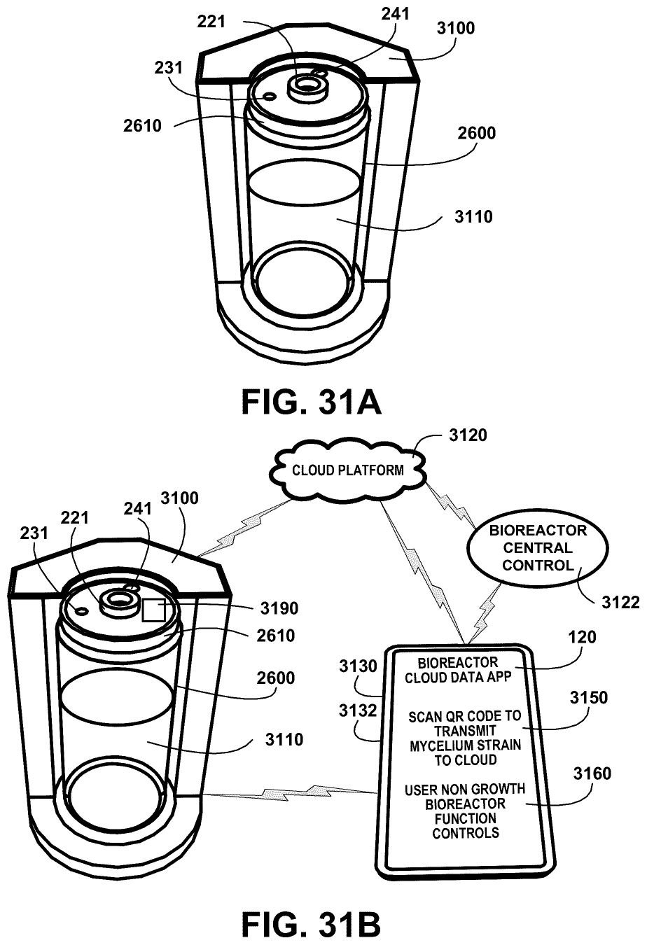

FIG. 31A shows for illustrative purposes only an example of refillable growing medium cartridge placed in bioreactor of one embodiment. FIG. 31A shows the refillable growing medium cartridge 2600 placed inside a bioreactor 3100. The cartridge cover 2610 includes a sterile port 231 for spore injection into the liquid nutrient 3110. The nutrient injection port 221 is used to add additional nutrients as needed during the cultivation process. Also present is a pressure relief valve 241 to prevent pressure build-up.

FIG. 31B shows for illustrative purposes only an example of a bioreactor app of one embodiment. FIG. 31B shows the refillable growing medium cartridge 2600, equipped with an aeration system to prevent spore clumping. Part B of the system is recyclable and can be returned for sterilization and refilling. The bioreactor 3100 cartridge cover 2610 includes the sterile port 231, nutrient injection port 221, and a pressure relief valve 241.

A user's smartphone 3130 has installed a bioreactor cloud data app 120 to allow the user to interact with the refillable growing medium cartridge 2600, the cloud platform 3120, and bioreactor central control 3122. The user's smartphone 3130 is used to scan QR code to transmit mycelium strain to cloud 3150. The user may operate with the bioreactor cloud data app 120 or user non growth bioreactor function controls 3160.

A QR code 3190 is attached to the cartridge cover, containing details about the mycelium strain. When scanned by the user's smartphone camera 3132, the QR code transmits information to a cloud platform 3120. The cloud platform 3120 sends control settings for the bioreactor, such as agitation rate, temperature, and oxygen flow, optimizing the environment for mycelium growth. The bioreactor cloud data app 120 also allows the user to control non-growth functions, for example, LED light color. The bioreactor central control 3122 generates the QR code for identifying cultured mycelium and sends batch progress updates to the user, making adjustments to the bioreactor as the culture develops.

In one embodiment, a portable and compact bioreactor for controlled cultivation of mycelium spores includes a bioreactor cartridge with an injection port, wherein the bioreactor cartridge is removably coupled to the bioreactor and contains a sterile substrate comprising organic materials as a nutrient, and wherein the injection port is a sealed, one-way sterile inlet configured to introduce the mycelium spores and predetermined nutrients into the sterile substrate liquid nutrient 3110 while preventing contamination.

Further including components comprising a pressure relief valve coupled to the bioreactor configured to prevent pressure build-up from heating the sterile substrate; a bioreactor microcontroller coupled to the bioreactor cartridge configured to monitor heat, oxygen, and nutrient levels; a heating element coupled to the bioreactor microcontroller configured to heat the sterile substrate within the bioreactor cartridge to a predetermined temperature; an air pump coupled to the bioreactor cartridge and to the bioreactor microcontroller configured to supply oxygenation of the mycelium spores; an agitator located within the bioreactor cartridge and coupled to a motor driver coupled to the bioreactor microcontroller and configured to rotate at a predetermined speed to distribute heat, oxygen, and nutrients among the mycelium spores with predetermined parameters; a cloud control system coupled to the bioreactor microcontroller configured to analyze empirical data related to cultivation of the mycelium spores to determine the predetermined speed, the predetermined parameters and the predetermined nutrients to produce specific harvest results and to make future automatic adjustments to the predetermined speed, the predetermined parameters and the predetermined nutrients based on the specific harvest results; and a bioreactor cloud data app coupled to a mobile device of the user configured to remotely monitor settings of the bioreactor, to allow the user to adjust the settings and to receive recommended settings based on the analysis of the cloud control system.

In yet another embodiment, the portable and compact bioreactor for controlled cultivation of mycelium spores is further comprising an artificial intelligence (AI) and machine learning bioreactor cloud control system wirelessly coupled to a bioreactor cloud data app coupled to a user's mobile device configured to remotely monitor settings of the bioreactor, to allow the user to remotely adjust the settings and to receive recommended settings based on the analysis of the cloud control system. Wherein the bioreactor cloud data app coupled to a user's mobile device is further configured to allow the user to self-report harvest results to the artificial intelligence (AI) and machine learning bioreactor cloud control system.

The bioreactor microcontroller bioreactor cloud data app is configured to receive parameter adjustment settings to automatically regulate parameter settings and optimize cultivation results in the bioreactor cartridge based on the analytical input data from the artificial intelligence (AI) and machine learning bioreactor cloud control system including user self-reported harvest results. Wherein the empirical data is transmitted automatically to the artificial intelligence (AI) and machine learning bioreactor cloud control system from each bioreactor microcontroller, user mobile device via the bioreactor cloud data app.

Further comprising the artificial intelligence (AI) and machine learning bioreactor cloud control system is further configured to compare past harvest results and past parameter settings to current harvest results and cultivation settings to determine optimal settings during cultivation that produce optimized harvest results to base recommended adjustments. Wherein the bioreactor includes a front pivoting section of a top cover section to pivot open for the installation of a bioreactor cartridge and make connections to the microcontroller, air pump oxygenation system, agitator drive and positioned above the heating element.

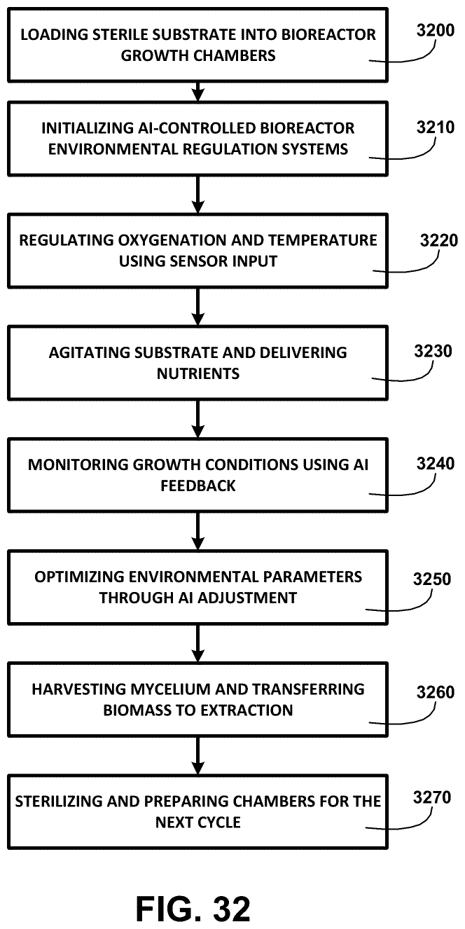

FIG. 32 shows a block diagram of an overview flow chart of AI-controlled mycelium cultivation manufacturing of one embodiment. FIG. 32 shows loading sterile substrate into bioreactor growth chambers 3200. Sterile, nutrient-rich substrate is loaded into sealed and sterilized growth chambers to initiate cultivation. Initializing AI-controlled bioreactor growth chambers environmental regulation systems 3210. The artificial intelligence control platform is initialized to manage and regulate all environmental parameters within the chambers.

Regulating oxygenation and temperature using sensor input 3220. Oxygen levels and temperature are actively regulated through sensor feedback to maintain optimal growth conditions. Agitating substrate and delivering nutrients 3230. The substrate is continuously agitated while nutrients are delivered at scheduled intervals to ensure uniform distribution and metabolic support. Monitoring growth conditions using AI feedback 3240. Sensor data is monitored in real time, and growth conditions are analyzed by the AI system for compliance with optimal profiles. Optimizing environmental parameters through AI adjustment 3250. The AI system adjusts environmental variables such as airflow, temperature, and agitation based on predictive analytics and historical data. Harvesting mycelium and transferring biomass to extraction 3260. Mature mycelium is harvested and transferred from the growth chambers to extraction systems for bioactive compound isolation. Sterilizing and preparing chambers for the next cycle 3270. The growth chambers are sterilized and reset to prepare for a subsequent cultivation cycle under aseptic conditions.

The large-scale mycelium cultivation system described herein is engineered for high-throughput commercial production of therapeutic compounds derived from mushroom mycelium. This system integrates advanced environmental regulation hardware, artificial intelligence (AI)-driven control algorithms, and cloud-based data analytics to enable a precision-controlled cultivation environment that supports consistent, year-round production. The cultivation framework is composed of multiple modular growth chambers designed to operate in concert under centralized digital oversight. These chambers are constructed using high-grade stainless steel, borosilicate glass, or biocompatible polymer materials, offering sterility, chemical resistance, and durability under repeated thermal sterilization cycles.

The growth chambers are arranged in linear or vertical stacks depending on available floor space, facility layout, and target production capacity. Each chamber operates semi-independently but remains integrated within a unified process control system. This modular architecture facilitates simultaneous cultivation of multiple fungal strains and supports scalability across both research and industrial applications. Environmental parameters—such as temperature, oxygen concentration, agitation rate, and nutrient availability—are monitored in real-time through a network of precision sensors embedded throughout the chamber structure. Each sensor transmits data to a microcontroller, which then relays the information to the cloud-based AI control system for analysis and directive generation.

Oxygenation is facilitated by a dedicated airflow system, comprising high-efficiency pumps and sterile air filters that supply each chamber with clean, regulated air. Oxygen is introduced into the substrate using sparging mechanisms such as air stones or microbubble diffusers. These devices promote even gas distribution and minimize hypoxic zones that could impede mycelial development. The agitation subsystem, which includes magnetically coupled impellers or direct-drive mixers, ensures uniform mixing of the substrate. This mixing facilitates homogenous distribution of nutrients, temperature, and oxygen, and also prevents sedimentation and compaction of the growth medium.

Temperature regulation is provided by externally mounted heating elements and optional cooling jackets. Each chamber contains an internal temperature sensor embedded in the substrate mass, which feeds data to the microcontroller. The AI system adjusts heating input dynamically based on growth phase and strain-specific thermal requirements. Standard operating temperatures range between 20° C. and 30° C., with precision control within ±0.5° C. to support consistent metabolic activity and biosynthesis of desired secondary metabolites.

The AI-driven control system is at the core of the cultivation platform. It leverages historical cultivation data, live sensor input, and predictive models to dynamically adjust chamber parameters. Nutrient feed schedules, oxygenation rates, and agitation profiles are automatically updated based on AI-identified patterns and environmental feedback. The cloud-based controller aggregates chamber-level performance data and compares it against known successful growth profiles, generating optimization protocols to increase biomass yield and bioactive compound concentration. This data-centric control approach ensures consistent quality, reduces biological waste, and improves overall process efficiency.

Chambers include aseptic access ports for nutrient infusion, liquid sampling, and harvest operations. These ports are designed to interface with automated liquid handling systems, allowing for sterile, hands-free process interventions. Once a chamber reaches optimal biomass density, the contents can be pumped directly to downstream extraction units where bioactive compounds are isolated using techniques such as solvent extraction, supercritical CO₂ extraction, and ultrasound-assisted methods. Chamber cleaning and sterilization cycles are then initiated in preparation for the next cultivation run.

Unlike conventional mushroom cultivation techniques that depend on soil, outdoor environments, or full fruiting body development, this system focuses on high-density mycelial growth under highly controlled conditions. By decoupling growth from environmental variability and enabling real-time process adaptation, the system achieves superior reproducibility and efficiency. Furthermore, the integrated AI infrastructure ensures that each cultivation cycle contributes new data to refine the operational parameters, fostering continuous process improvement and adaptive learning at scale.

In summary, the large-scale AI-controlled mycelium cultivation system offers a robust, scalable, and intelligent solution for commercial production of mushroom-derived medicinal compounds. It provides precision control of critical growth conditions, reduces operational labor, eliminates contamination risks, and maximizes production efficiency. Designed to comply with regulatory standards and suitable for pharmaceutical, nutraceutical, and biotechnological applications, this system advances the industrial cultivation of fungal biomass into a repeatable, data-optimized manufacturing process.

The foregoing has described the principles, embodiments, and modes of operation of the present invention. However, the invention should not be construed as being limited to the particular embodiments discussed. The above-described embodiments should be regarded as illustrative rather than restrictive, and it should be appreciated that variations may be made in those embodiments by workers skilled in the art without departing from the scope of the present invention as defined by the following claims.

What is claimed is:

1. A method for cultivating mycelium in a bioreactor controlled environment, comprising:
loading a sterile growth substrate into a plurality of sealed growth chambers;
initializing an artificial intelligence (AI)-controlled cultivation system to manage environmental parameters within each growth chamber;
regulating oxygenation levels within the growth chambers via sterile air delivery and microbubble diffusers, based on sensor feedback;
applying thermal regulation to each growth chamber by activating heating elements, wherein temperature setpoints are controlled by the AI system based on real-time and historical data;
activating internal agitation mechanisms to uniformly distribute oxygen, nutrients, and heat throughout the substrate;
monitoring growth conditions including temperature, oxygen concentration, and agitation speed, and transmitting sensor data to the AI-controlled cultivation system;
dynamically adjusting one or more environmental parameters based on analysis performed by the AI-controlled cultivation system to optimize mycelial growth;
harvesting mycelial biomass from the growth chambers upon achieving a predetermined growth threshold; and
sterilizing and resetting the growth chambers for a subsequent cultivation cycle.

2. The method of claim 1, wherein the growth chambers are constructed of stainless steel, glass, or polymeric material and configured for high-temperature sterilization.

3. The method of claim 1, wherein the AI-controlled cultivation system utilizes a machine learning algorithm trained on historical cultivation data to generate parameter adjustments.

4. The method of claim 1, further comprising injecting liquid nutrients into the substrate through aseptic fluid delivery ports at defined intervals.

5. The method of claim 1, wherein the microbubble diffusers generate bubbles within a range of 0.5 μm to 5 μm to improve oxygen dissolution in the substrate.

6. The method of claim 1, wherein the internal agitation mechanism comprises a magnetically coupled impeller configured to rotate at a speed dynamically regulated by the AI system.

7. The method of claim 1, wherein harvested mycelial biomass is transferred to an extraction unit for isolation of bioactive compounds using solvent or supercritical CO₂ extraction.

8. A bioreactor system for cultivating mycelium in a controlled environment, comprising:
a plurality of growth chambers each containing a substrate region for cultivating mycelium;
an artificial intelligence (AI)-controlled cultivation system configured to regulate environmental parameters;
at least one oxygen delivery system comprising a sterile air source and a microbubble diffuser operatively coupled to each growth chamber;
a thermal regulation assembly comprising a heating element and temperature sensor associated with each growth chamber;
an agitation mechanism operatively positioned within each growth chamber; and
a control unit configured to receive sensor data and transmit AI-generated instructions to adjust environmental conditions within each growth chamber.

9. The bioreactor system of claim 8, wherein each growth chamber includes an aseptic access port for nutrient delivery and sample collection.

10. The bioreactor system of claim 8, wherein the AI-controlled cultivation system is wirelessly coupled to a remote database and cloud analytics platform.

11. The bioreactor system of claim 8, wherein the agitation mechanism comprises a motor-driven shaft with paddles or impellers adapted to operate within the substrate medium.

12. The bioreactor system of claim 8, wherein the thermal regulation assembly maintains temperature within +0.5° C. of a target setpoint using feedback from the AI control unit.

13. The bioreactor system of claim 8, further comprising a feedback interface configured to compare active growth parameters to historical performance data and automatically adjust operation.

14. The bioreactor system of claim 8, wherein the control unit is further configured to initiate a sterilization sequence after harvest completion.

15. A mycelium cultivation and extraction bioreactor system, comprising:
a plurality of sealed growth chambers containing a nutrient substrate;
an AI-controlled environmental regulation system configured to manage temperature, oxygenation, and agitation conditions within the growth chambers;
a fluidic control subsystem configured to inject nutrients and withdraw liquid samples;
a harvesting module operatively coupled to each growth chamber and configured to transfer biomass;
an integrated extraction unit configured to isolate bioactive compounds from harvested mycelium; and
a data analytics engine configured to generate cultivation optimization instructions based on real-time and historical data.

16. The bioreactor system of claim 15, wherein the extraction unit comprises one or more of: solvent extraction, ultrasound-assisted extraction, or supercritical CO₂ extraction modules.

17. The bioreactor system of claim 15, wherein the harvesting module includes sterile fluid transfer conduits and programmable valves.

18. The bioreactor system of claim 15, wherein the AI-controlled environmental regulation system adjusts conditions in response to predicted growth trajectories based on prior cycles.

19. The system of claim 15, further comprising a sterilization subsystem configured to purge and reset each growth chamber following harvest.

20. The bioreactor system of claim 15, wherein the fluidic control subsystem comprises aseptic ports and peristaltic pumps for controlled media flow.
